(19) United States
(12) Patent Application Publication
(10) Pub. No.: US 2026/0183488 A1
(43) Pub. Date: Jul. 2, 2026

(54) A SUB-ASSEMBLY FOR A MEDICAMENT DELIVERY DEVICE

(71) Applicant: SHL Medical AG, Zug (CH)

(72) Inventors: Joakim Lindholm, Nacka Strand (SE); Camilla Gibson, Nacka Strand (SE)

(21) Appl. No.: 19/133,921

(22) PCT Filed: Dec. 5, 2023
(86) PCT No.: PCT/EP2023/084273

§ 371 Date: May 29, 2025

(30) Foreign Application Priority Data
Dec. 5, 2022 (EP) 22211540.4

(51) Int. Cl. A61M 5/32; A61M 5/20; A61M 5/315

(52) U.S. Cl. CPC A61M 5/3202; A61M 5/31501; A61M 5/31571; A61M 5/3234; A61M 2005/2073

(57) ABSTRACT

The present disclosure provides a sub-assembly for a medicament delivery device, the sub-assembly extending along an axis from a proximal end to a distal end. The sub-assembly includes a housing, the housing being tubular and comprising a flexible arm, the flexible arm comprising a protrusion extending towards the axis and a surface facing away from the axis. The sub-assembly further includes a cap, the cap being removably attached to a proximal end of the housing, the cap comprising a surface facing towards the axis. The sub-assembly further includes a syringe holder attached to the housing, the syringe holder comprising a protrusion extending away from the axis; a medicament delivery member guard arranged telescopically at least partially inside the housing, the medicament delivery member guard comprising a recess or cut-out. The surface of the cap is adjacent to the surface of the housing to restrict movement of the flexible arm away from the axis when the cap is attached to the housing. The protrusion of the flexible arm is in the recess or cut-out of the medicament delivery member guard. The protrusion of the syringe holder is adjacent to the protrusion of the flexible arm of the housing, so that, when the cap is attached to the housing, the protrusion of the flexible arm of the housing

### TECHNICAL FIELD

The application concerns sub-assemblies for medicament delivery devices.

### BACKGROUND

Medicament delivery devices such as autoinjectors have been developed to help simplify drug injection, including enabling self-injection by users that are not medical professionals. Whilst sophisticated devices such as those in WO2022/078986 and WO2022/078987 are known, the applicant has appreciated that there are nevertheless further improvements that could be made to these existing designs.

### SUMMARY

Reference should now be made to the appended claims.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

An aspect concerns a lock component for a medicament delivery device, the lock component extending along an axis from a proximal end to a distal end, the lock component comprising a base, a pivot and a flexible arm, wherein the flexible arm is attached to the base by the pivot, wherein the flexible arm comprises a proximal part extending proximally from the pivot, wherein the flexible arm is configured to support accidental activation prevention in a medicament delivery device, and wherein the flexible arm is configured to support medicament delivery member lockout in a medicament delivery device. Although it may be counterintuitive to design a more complicated component (for example due to the challenge of moulding a more complex component), this can reduce the number of components compared to existing solutions.

Optionally, the flexible arm comprises a first proximally facing surface that is configured to engage a distally facing surface of another medicament delivery device component so as to provide accidental activation prevention in a medicament delivery device. Optionally, the lock component comprises a first protrusion, and the first proximally facing surface is on the first protrusion. Optionally, the proximal part of the flexible arm comprises the first proximally facing surface. Optionally, the first protrusion extends away from the axis. Optionally, the proximally facing surface extends perpendicular to the axis.

Optionally, the flexible arm comprises a second proximally facing surface that is configured to engage a distally facing surface of another medicament delivery device component so as to provide a medicament delivery member guard lockout after medicament delivery device use. Optionally, the flexible arm comprises a second protrusion, and wherein the second protrusion comprises the second proximally facing surface. Optionally, the second protrusion extends from the proximal part of the flexible arm. Optionally, the second protrusion extends away from the axis relative to the rest of the proximal part of the flexible arm. Optionally, the second proximally facing surface comprises a notch that is configured to engage said distally facing surface of another medicament delivery device component so as to provide a medicament delivery member guard lockout after medicament delivery device use. Optionally, the notch is v- or u-shaped. Optionally, the second protrusion is the proximal-most part of the proximal part of the flexible arm

Optionally, the first protrusion is aligned in the axial direction with the second protrusion. Optionally, the first protrusion is spaced apart from the second protrusion. Optionally, the first protrusion is arranged distal to the second protrusion.

Optionally, the lock component comprises a snap-fit hole to attach the lock component to a medicament delivery container holder. Optionally, the lock component comprises an arm to lock the lock component to a housing. Optionally, the lock component comprises either a screw thread or a protrusion, so as to engage a corresponding screw thread on a plunger rod. Optionally, the lock component comprises a lock component arm, the lock component arm being flexible and comprising at least one tooth to engage a corresponding tooth on a driver of a medicament delivery device so as to limit rotation of the driver relative to the lock component prior to medicament delivery.

Optionally, the flexible arm is configured to support accidental activation prevention in a medicament delivery device by engaging a proximally facing surface of a lock activation sleeve of the medicament delivery device, thereby blocking distal movement of the lock activation sleeve relative to the flexible arm so as to stop medicament delivery device activation.

Optionally, the flexible arm is configured to support medicament delivery member lockout in a medicament delivery device by engaging a recess or slit in a housing of a medicament delivery device when a medicament delivery member guard of the medicament delivery device is pushed against the flexible arm, thereby blocking distal movement of the medicament delivery member guard relative to the flexible arm.

Optionally, the flexible arm comprises a distal part extending distally from the pivot. Optionally, the lock component is a single integral part.

An aspect concerns a medicament delivery device sub-assembly comprising a lock activation sleeve and any lock component wherein the flexible arm comprises a first proximally facing surface that is configured to engage a distally facing surface of another medicament delivery device component so as to provide accidental activation prevention in a medicament delivery device, wherein the another medicament delivery device component is the lock activation sleeve. Optionally, the lock activation sleeve comprises a pad. Optionally, the pad is adjacent to the distal part of the flexible arm of the lock component. Optionally, the lock activation sleeve comprises a base and an arm extending from the base, and wherein the pad is an inwardly facing surface of the arm. Optionally, the distally facing surface is in a recess or cut-out in the arm. Optionally, the lock activation sleeve extends around the lock component.

Optionally, the sub-assembly comprises a medicament delivery member guard, and wherein a distally facing surface of the medicament delivery member guard is configured to engage a corresponding proximally facing surface of the lock activation sleeve so as to move the lock activation sleeve in a distal direction relative to the lock component, thereby allowing medicament delivery.

An aspect concerns a medicament delivery device sub-assembly, wherein the sub-assembly comprises a housing and any lock component wherein the flexible arm comprises a second proximally facing surface that is configured to engage a distally facing surface of another medicament delivery device component so as to provide a medicament delivery member guard lockout after medicament delivery device use, and wherein the another medicament delivery device component is the housing. Optionally, the housing comprises a recess or cut-out, and wherein the second protrusion is at least partially inside the recess or cut-out after the medicament delivery device is used. Optionally, the lock component is inside the housing.

Optionally, the sub-assembly comprises a medicament delivery container holder. Optionally, the sub-assembly comprises a driver. Optionally, the sub-assembly comprises a plunger rod.

An aspect concerns a medicament delivery device powerpack comprising any lock component as described above or any sub-assembly as described above.

An aspect concerns a medicament delivery device comprising any lock component as described above or any sub-assembly as described above or any powerpack as described above. Optionally, the medicament delivery device is an autoinjector.

An aspect concerns a medicament delivery device component, the component comprising a first flexible arm, the first flexible arm being configured to prevent accidental activation of a medicament delivery device and the first flexible arm being configured to provide a lockout after use of said medicament delivery device so that a medicament delivery member of said medicament delivery device is protected.

An aspect concerns a sub-assembly for a medicament delivery device, the sub-assembly extending along an axis from a proximal end to a distal end, the sub-assembly comprising: a housing, the housing being tubular and comprising a flexible arm, the flexible arm comprising a protrusion extending towards the axis and a surface facing away from the axis; a cap, the cap being removably attached to a proximal end of the housing, the cap comprising a surface facing towards the axis; a syringe holder attached to the housing, the syringe holder comprising a protrusion extending away from the axis; a medicament delivery member guard arranged telescopically at least partially inside the housing, the medicament delivery member guard comprising a recess or cut-out; wherein the surface of the cap is adjacent to the surface of the housing to restrict movement of the flexible arm away from the axis when the cap is attached to the housing; wherein the protrusion of the flexible arm is in the recess or cut-out of the medicament delivery member guard; and wherein the protrusion of the syringe holder is adjacent to the protrusion of the flexible arm of the housing, so that, when the cap is attached to the housing, the protrusion of the flexible arm of the housing and the protrusion of the syringe holder together restrict distal movement of the medicament delivery member guard relative to the housing. An aspect concerns a medicament delivery device comprising the sub-assembly. Optionally, the medicament delivery device is an autoinjector.

Optionally, the flexible arm of the housing comprises an outwardly extending protrusion, and the outwardly extending protrusion comprises the surface facing away from the axis.

Optionally, the cap comprises a protrusion, and the surface facing towards the axis is on the protrusion. Optionally, the cap comprises a surface facing away from the axis; wherein the surface facing away from the axis is adjacent to an inwardly facing surface of the housing, thereby restricting movement of the protrusion of the cap away from the axis. Optionally, the syringe holder is tubular. Optionally, the sub-assembly comprises a medicament delivery member guard spring arranged between the housing and the medicament delivery member guard to bias the medicament delivery member guard in the proximal direction relative to the housing. Optionally, the housing comprises a proximal housing and a distal housing, and the proximal housing and the distal housing are two separate components. Optionally, the distal housing comprises the inwardly facing surface of the housing and the proximal housing comprises the flexible arm of the housing. Optionally, the cap comprises a medicament delivery member shield remover. Optionally, the cap comprises the medicament delivery member shield remover, a cap housing and a cap insert, and the medicament delivery member shield remover is arranged between the cap housing and the cap insert. Optionally, an inner surface of the housing comprises a recess that can receive the flexible arm after the cap has been removed from the sub-assembly.

An aspect concerns a sub-assembly for a medicament delivery device, the sub-assembly extending along an axis from a proximal end to a distal end, the sub-assembly comprising: a housing, the housing being tubular and comprising a flexible arm, the flexible arm comprising a protrusion extending towards the axis and a surface facing away from the axis; a cap, the cap being removably attached to a proximal end of the housing, the cap comprising a surface facing towards the axis; a syringe holder attached to the housing, the syringe holder comprising a protrusion extending away from the axis; a medicament delivery member guard arranged telescopically at least partially inside the housing, the medicament delivery member guard comprising a recess or cut-out; wherein the surface of the cap is adjacent to the surface of the housing to restrict movement of the flexible arm away from the axis when the cap is attached to the housing; wherein the protrusion of the flexible arm is in the recess or cut-out of the medicament delivery member guard; and wherein the protrusion of the syringe holder is aligned in a radial direction relative to the axis with the protrusion of the flexible arm of the housing, so that, when the cap is attached to the housing, the protrusion of the flexible arm of the housing and the protrusion of the syringe holder together restrict distal movement of the medicament delivery member guard relative to the housing.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

FIGS. 1 and 2 show perspective views of a lock component.

FIGS. 3 and 4 show side views of the lock component of FIG. 1.

FIGS. 5 and 6 show end views of the lock component of FIG. 1.

FIG. 7 shows another perspective view of the lock component of FIG. 1.

FIG. 8 shows a cross-section view of part of a medicament delivery device comprising the lock component of FIG. 1, showing the initial position before use.

FIG. 9 shows a cross-section view of part of the medicament delivery device of FIG. 8, after the needle guard has been moved in the distal direction.

FIG. 10 shows a cross-section view of part of the medicament delivery device of FIG. 8 after injection.

FIG. 11 shows a cross-section view of part of the medicament delivery device of FIG. 8, when an attempt has been made to push the needle guard back in the distal direction after the lock created by the lock component 101 has been set.

FIG. 12 shows a perspective view of a medicament delivery device according to FIGS. 1 to 11.

FIG. 13 shows an exploded perspective view of the medicament delivery device of FIG. 12.

FIG. 14 shows a perspective view of the lock activation sleeve of the medicament delivery device of FIG. 12.

FIG. 15 shows a perspective view of the driver of the medicament delivery device of FIG. 12.

FIG. 16 shows a perspective view of the powerpack lock, the plunger rod, the spinner and the spinner cap of the medicament delivery device of FIG. 12.

FIGS. 17 and 18 show perspective views of the thrust bearing of the medicament delivery device of FIG. 12.

FIG. 19 shows a perspective view of the syringe holder of the medicament delivery device of FIG. 12.

FIG. 20 shows a perspective exploded view of the cap of the medicament delivery device of FIG. 12.

FIG. 21 shows a perspective view of the syringe of the medicament delivery device of FIG. 12, including a rigid needle shield 364 and a flange 365.

FIG. 22 shows a perspective view of the needle guard of the medicament delivery device of FIG. 12.

FIG. 23 shows a perspective view of the powerpack housing of the medicament delivery device of FIG. 12.

FIG. 24 shows a perspective view of the proximal housing of the medicament delivery device of FIG. 12.

FIG. 25 shows a cross-section view of a proximal portion of the medicament delivery device of FIG. 12.

FIGS. 26 to 28 show views of part of an example syringe holder.

FIGS. 29 to 30 show views of part of another example syringe holder.

FIG. 31 shows part of another example syringe holder.

FIG. 32 shows part of another example syringe holder.

FIG. 33 shows part of another example syringe holder and an adjacent part of an example housing.

FIG. 34 shows part of another example syringe holder.

FIGS. 35 and 37 show views of the example syringe holder of FIG. 29.

FIGS. 36 and 38 show views of another example syringe holder.

FIGS. 39 to 42 each show an example of a distal end of another example syringe holder.

FIG. 43 shows a proximal end of another example syringe holder.

FIG. 44 shows a proximal end of another example syringe holder.

FIG. 45 shows a cross-section view of part of a medicament delivery device.

FIG. 46 shows a cross-section view of part of another medicament delivery device.

FIG. 47 shows a perspective view of a proximal housing.

FIG. 48 shows a perspective view of the proximal housing of FIG. 24.

FIGS. 49 and 50 show close-up views of example proximal housing flexible ribs.

FIG. 51 shows a perspective view of part of a needle guard.

FIGS. 52 and 53 show views of the needle guard of FIG. 22.

FIG. 54 shows a perspective view of part of another needle guard.

FIGS. 55 and 56 show views of part of a cap housing.

FIGS. 57 and 58 show views of part of another cap housing.

FIG. 59 shows a cross-section view of part of a medicament delivery device.

FIG. 60 shows a cross-section view of part of another medicament delivery device.

FIG. 61 shows a perspective view of a cap housing.

FIG. 62 shows a perspective view the cap housing of FIG. 20.

FIG. 63 shows a cross-section perspective view of another cap housing.

FIG. 64 shows a partially see-through perspective view of part of the cap housing 162 of FIG. 63.

FIGS. 65 and 66 show views of part of a medicament delivery device.

FIG. 67 shows the cap insert of the medicament delivery device of FIG. 65.

FIG. 68 shows an example of a cap insert.

FIG. 69 shows an example of another cap insert.

FIG. 70 shows an example of another cap insert.

FIG. 71 shows another view of the cap insert of FIG. 20.

FIGS. 72 to 74 show different views of a driver.

FIG. 75 shows part of another driver.

FIGS. 76 and 77 show different views of another driver.

FIGS. 78 and 79 show different views of another driver.

FIG. 80 shows a powerpack housing.

FIGS. 81 and 82 show views of another powerpack housing.

FIG. 83 shows another powerpack housing.

FIG. 84 shows another powerpack housing.

FIG. 85 shows another powerpack housing.

FIGS. 86, 87 and 90 show views of a powerpack lock.

FIGS. 88, 89 and 91 show views of another powerpack lock.

FIGS. 92 to 94 show various powerpack lock examples.

FIG. 95 shows another view of the powerpack lock of FIG. 16.

FIG. 96 shows another powerpack lock.

FIG. 97 shows another view of the powerpack lock of FIG. 95.

FIGS. 98 and 99 show views of another powerpack lock.

FIGS. 100 and 102 show parts of a plunger rod.

FIG. 101 shows part of another plunger rod.

FIG. 103 shows an example of the structure of the spinner of FIG. 16.

FIGS. 104 and 107 show an example of the structure of the spinner of FIG. 16.

FIGS. 105 and 106 show an example of the structure of the spinner of FIG. 16.

FIG. 108 shows another perspective view of the lock activation sleeve of FIG. 14.

FIGS. 109 to 111 show three alternative lock activation sleeves.

FIGS. 112 and 113 show perspective views of another alternative lock component.

FIGS. 114 and 115 show views of another alternative lock component.

FIGS. 116 to 124 show various views of a distal housing, thereby showing a number of alternative structures for the distal housing.

FIGS. 125 and 126 show part of two further embodiments of the lock component.

FIG. 127 shows a cross-section view of a proximal portion of a medicament delivery device of a further embodiment.

FIG. 128 shows an integrated housing and syringe carrier of the embodiment of FIG. 127.

### DETAILED DESCRIPTION

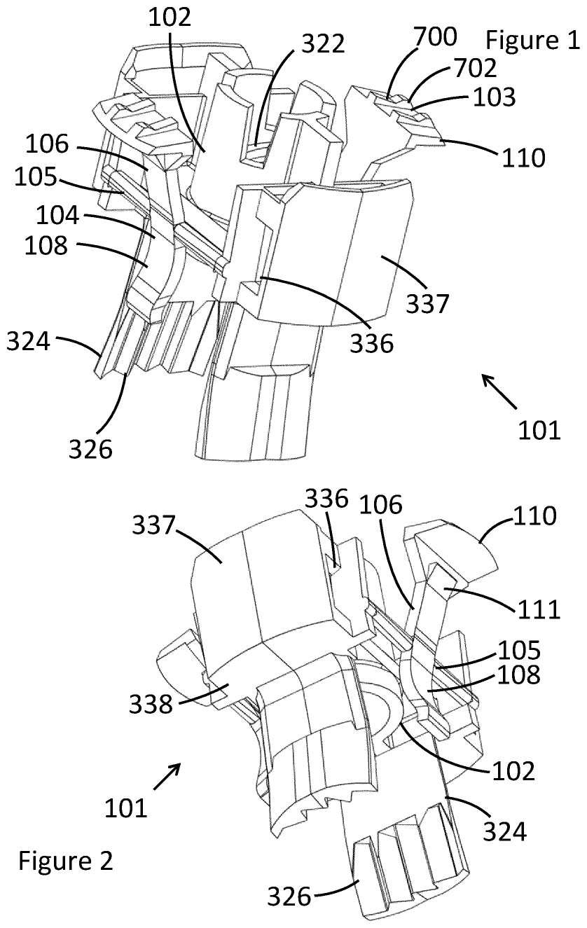

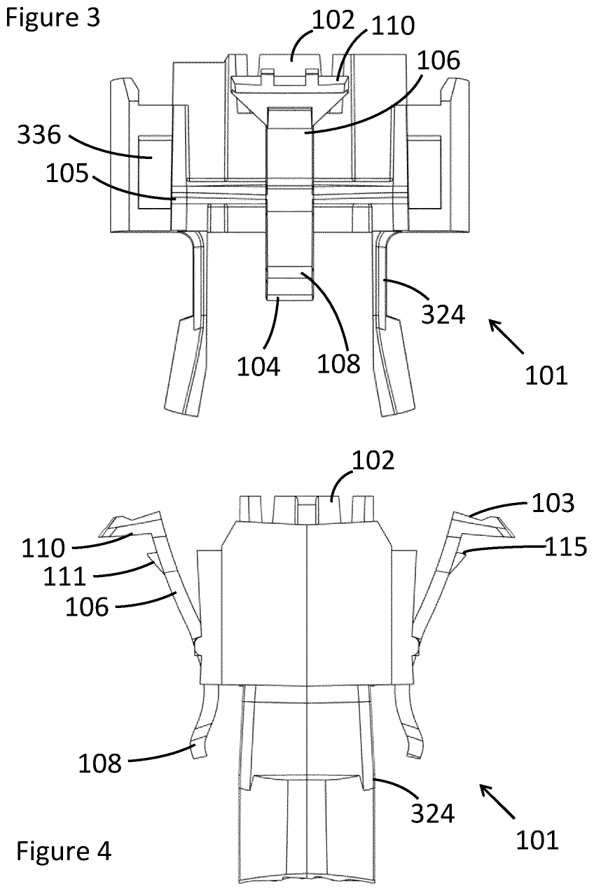

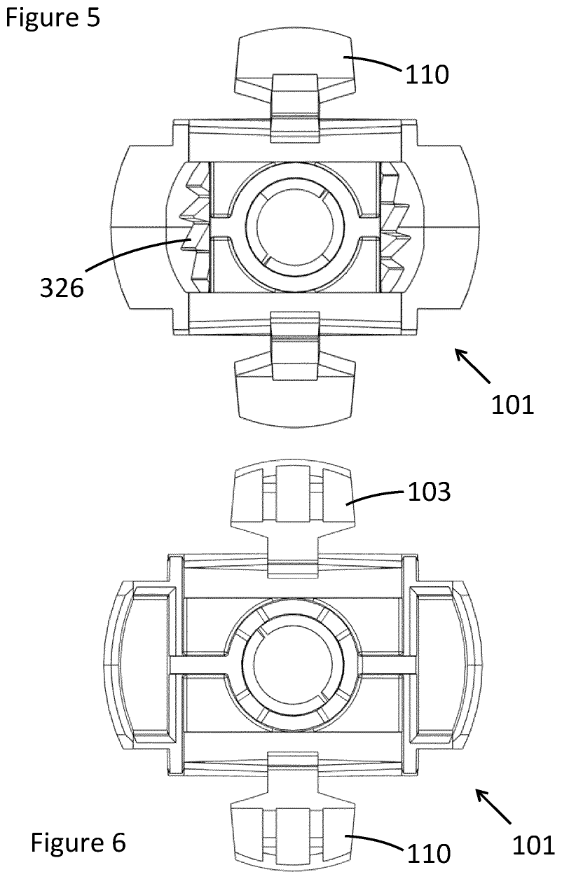

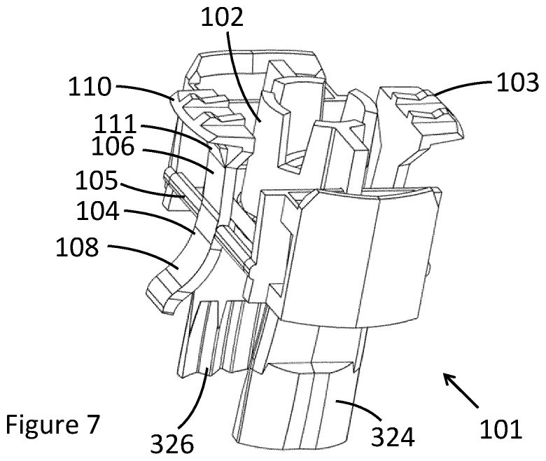

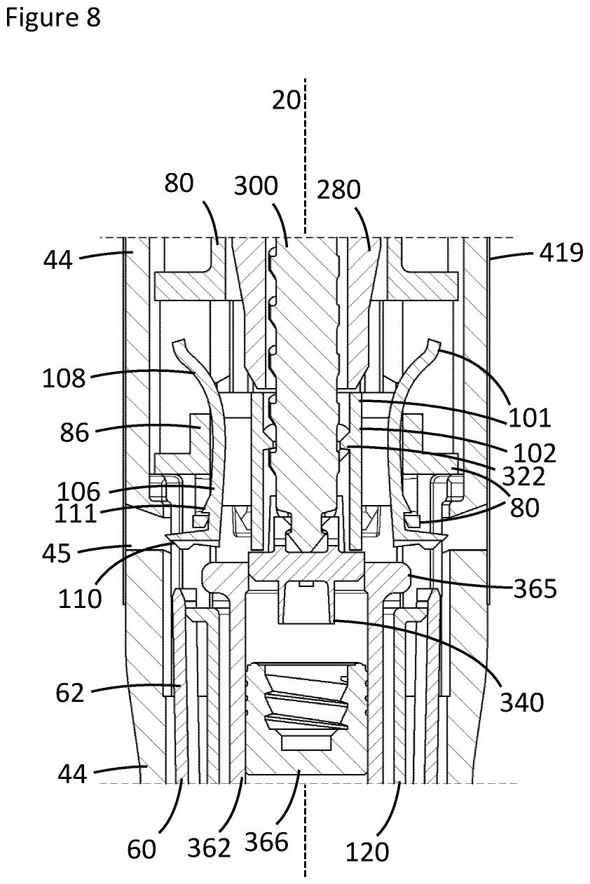

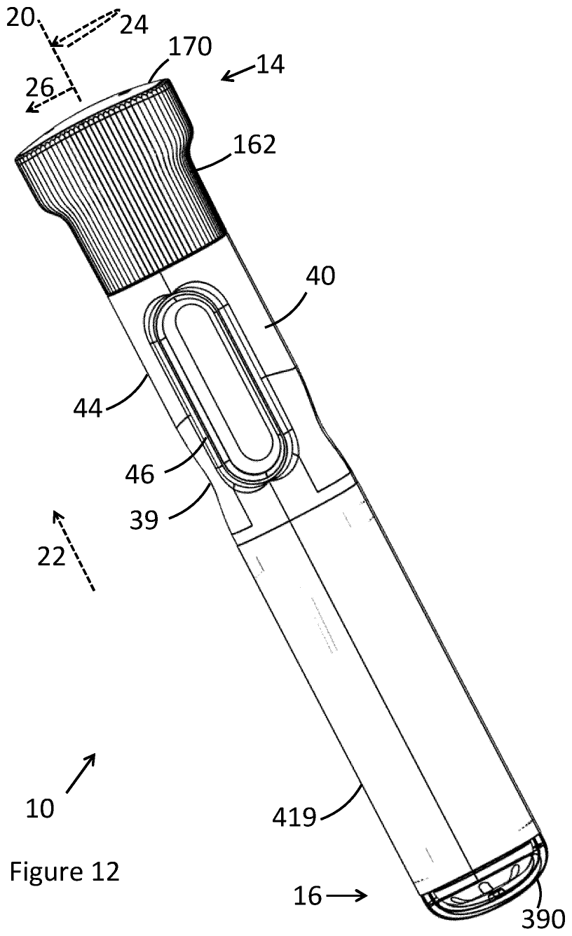

A lock component 101 is shown in FIGS. 1 to 7. The lock component 101 is for a medicament delivery device such as an autoinjector 10. The lock component extends along an axis 20 (see FIG. 12, for example) from a proximal end to a distal end. The lock component comprises a base 102, a flexible arm 104 and a pivot 105. The flexible arm 104 is attached to the base 102 by the pivot 105. The flexible arm comprises a proximal part 106 extending proximally from the pivot 105 (and optionally a distal part 108 extending distally from the pivot 105). In general, the flexible arm 104 is configured to support accidental activation prevention in a medicament delivery device such as an autoinjector, and the flexible arm is also configured to support medicament delivery member lockout in a medicament delivery device such as an autoinjector. Further description of how a lock component works within an example medicament delivery device is outlined below with reference to FIGS. 8 to 11 in particular.

The specific structure of the lock component can be altered in numerous ways, many of which will be discussed herein. The example shown in FIGS. 1 to 7 will now be described in more detail. The flexible arm 104 comprises a proximally facing surface 115 (see FIG. 4) that is configured to engage a distally facing surface of another medicament delivery device component lock activation sleeve 80 in this example, as described in more detail below) so as to provide accidental activation prevention in a medicament delivery device. More specifically, the proximally facing surface 115 can interact with a distal part of the lock activation sleeve 80 (in this example a distally facing surface 89 of the arm 84 of the lock activation sleeve 80) to help restrict unintentional movement in the distal direction of the lock activation sleeve prior to injection. In this particular example, the proximally facing surface 115 can also help avoid accidental activation even if a medicament delivery member guard such as the needle guard 60 (FIG. 22) is independently locked (for example by a cap) to avoid accidental activation. This is because the lock activation sleeve 80, which in this example is a separate component from the needle guard 60, could still be moved inside the device without the needle guard 60 moving, for example by accidentally dropping the device. As such, it can be beneficial to lock the lock activation sleeve 80 (and optionally also to lock the needle guard 60). An alternative solution could be to provide a spring that biases the lock activation sleeve 80 in the proximal direction.

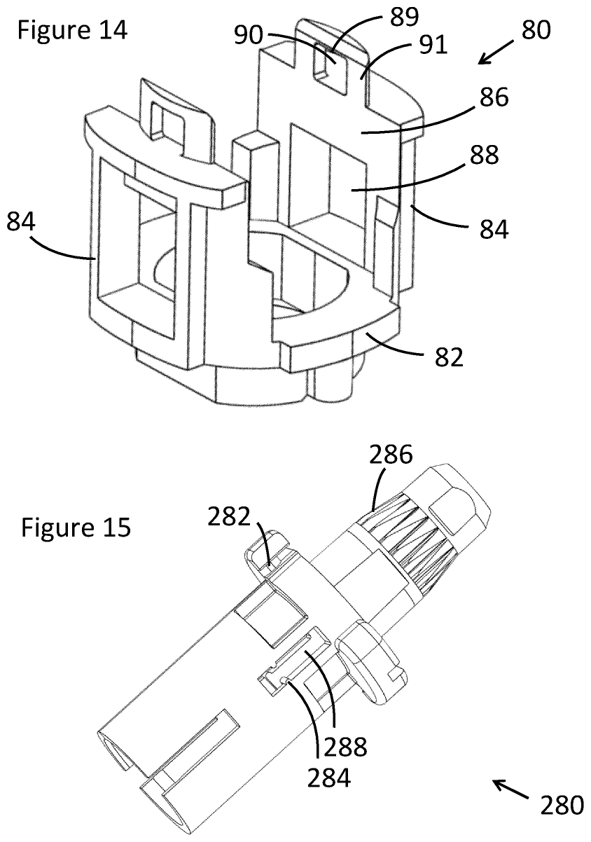

The proximally facing surface 115 is on a protrusion 111 (first protrusion) in this example, although the proximally facing surface could alternatively be part of the surface of a recess or cut-out instead. The protrusion 111 extends away from the axis in this example, and the proximal part 106 of the flexible arm 104 comprises the protrusion in this example. The proximally facing surface 115 extends perpendicular to the axis (and is planar in this example) in an assembled (completed) medicament delivery device, although the surface could alternatively extend at an angle of less than 90 degrees from the axis and could be non-planar. In this example, the protrusion 111 and the protrusion 110 are arranged so that the arm 91 of the lock activation sleeve 80 that comprises the distally facing surface 89 is arranged directly between the protrusion 111 and the protrusion 110 (see e.g. FIG. 8) in an assembled medicament delivery device, although this is not essential. In general, the particular shape of the protrusion 111 and the corresponding distally facing surface 89 of the lock activation sleeve 80 (in this example, the distally facing surface 89 is part of the wall of a second cut-out 90 of the lock activation sleeve 80, which is a cut-out 90 of the arm 91 of the lock activation sleeve 80, the arm 91 being part of the lock activation sleeve arm 84, see FIG. 14) is optional.

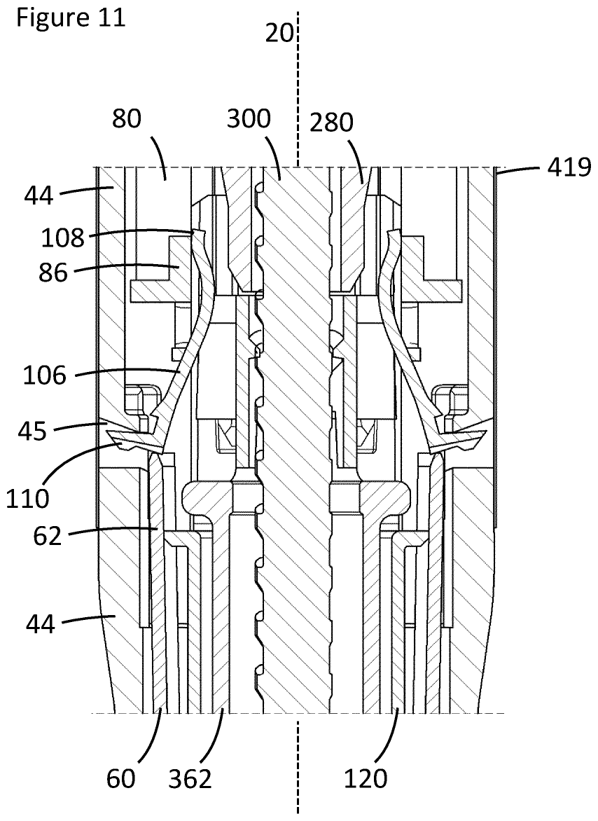

The flexible arm 104 comprises a second proximally facing surface 103 that is configured to engage a distally facing surface of another medicament delivery device component (a medicament delivery member guard in this example, or more specifically the needle guard 60 as shown in FIG. 11) so as to provide a medicament delivery member guard lockout after medicament delivery device use (this can be useful to hide the medicament delivery member after use and/or to prevent accidental needle stick injuries). The second proximally facing surface could simply be on the proximal end of the flexible arm, but in this example, the flexible arm 104 comprises a lockout protrusion 110 (second protrusion), and wherein the lockout protrusion 110 comprises the second proximally facing surface 103. In this example, the second protrusion 110 extends from the proximal part 106 of the flexible arm 104. In this example, the second protrusion extends away from the axis from the (rest of) the proximal part of the flexible arm, although this is optional and the protrusion could alternatively extend towards the axis. The second protrusion 110 is the proximal-most part of the proximal part of the flexible arm, although the second protrusion could alternatively be spaced apart from the proximal-most part of the flexible arm (in the axial direction). Optionally, the second proximally facing surface comprises a notch 700 that is configured to engage the distally facing surface of another medicament delivery device component (a medicament delivery member guard in this example, or more specifically the needle guard 60 as shown in FIG. 11) so as to provide a medicament delivery member guard lockout after medicament delivery device use. Optionally, as shown in FIG. 1 for example, the notch 700 is in a rib 702, with the rib 702 extending in the proximal direction from the protrusion 110 (in this example, the protrusion 110 primarily extends perpendicular to the axis). The notch can be v- or u-shaped, for example, and can help ensure that the needle guard arm 62 of the needle guard 60 does not slip past the protrusion 110, as the distal-most portion of the needle guard arm 62 engages the notch if the needle guard arm 62 is moved in the distal direction relative to the lock component after use of the medicament delivery device.

In addition to the aspects listed above, the lock component 101 also contains a number of further aspects in this example, though these aspects could typically alternatively be provided by another component of the medicament delivery device, such as a housing component (e.g. a housing, a distal housing, a proximal housing, and/or a powerpack housing) or a syringe holder.

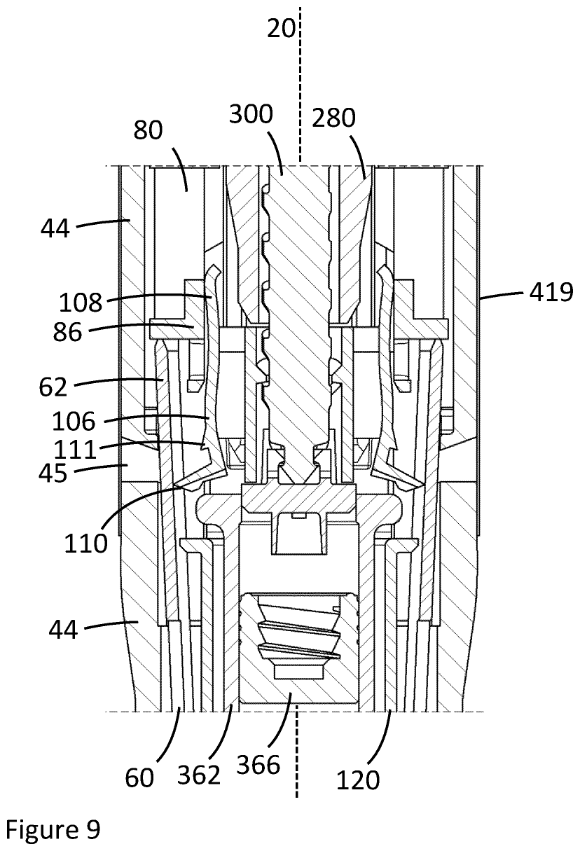

The lock component 101 comprises a lock component arm 324 (which is flexible). The lock component arm 324 comprises lock component teeth 326 (or in general at least one tooth). The lock component teeth 326 are configured to engage with driver teeth 286 to limit rotation of the driver 280 (described in more detail below) prior to medicament delivery device activation. This is achieved by the lock activation sleeve 80 limiting radial movement of the lock component arm 324 away from the axis in an assembled medicament delivery device. Axial movement of the lock activation sleeve 80 relative to the lock component 101 during device activation (as outlined in more detail below with reference to FIGS. 8 and 9 in particular) removes the radial movement restriction on the lock component arm 324, thereby allowing the lock component arm 324 to flex outwards relative to the driver teeth 286 and allowing the driver 280 to rotate relative to the lock component 101.

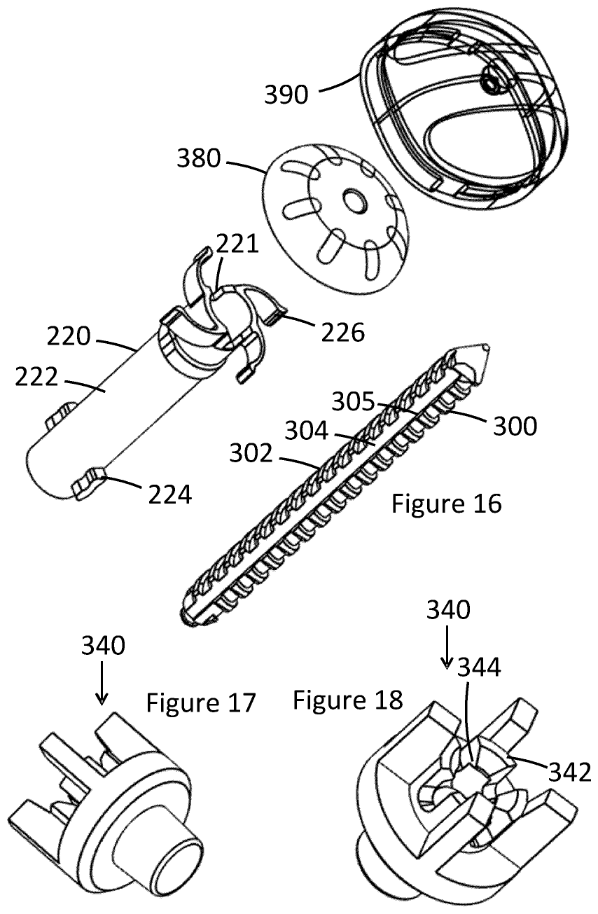

The lock component 101 comprises a lock component screw thread 322 that engages a corresponding plunger rod screw thread 302 (see FIG. 16, for example). When the driver 280 rotates the plunger rod 300 during medicament delivery (as described in more detail below when describing the plunger rod and driver in more detail), the plunger rod is rotated relative to the lock component 101, resulting in the plunger rod moving in the proximal direction relative to the lock component 101 due to the engagement between the plunger rod screw thread 302 and the lock component screw thread 322. The provision of a screw thread on both the plunger rod and the lock component may be beneficial, for example to provide a robust connection between the lock component and the plunger rod (which may be particularly useful when a high force is needed, for example with a viscous drug). However, a screw thread on one of the plunger rod and the lock component along with a protrusion on the other of the plunger rod and the lock component could alternatively be provided.

Alternatively, the screw (or protrusion) on the lock component could be removed, with the feature provided on another component (such as the powerpack housing 240 or the syringe holder 120) instead.

The lock component 101 comprises a snap-fit hole 336, which can be used to attach the lock component 101 to the syringe holder 120. Alternatively or additionally, the syringe holder can be held in place within the device by attachment to a different part of the lock component, and/or by attachment to other medicament delivery member device components such as a housing. The lock component comprises an arm 337, which is used to lock the lock component to the housing (specifically to the distal housing in the example shown in FIG. 12); this can keep the lock component in place relative to the housing. However, the lock component does not need to be directly attached to a specific housing part.

Two examples of alternative lock components are described below with reference to FIGS. 112 to 115.

With reference to FIGS. 8 to 11, the relative movement during use of some of the parts around the lock component 101 will now be described. FIG. 8 shows the initial position before use (and before activation) of the autoinjector. This is the position in which the autoinjector would typically be kept in between final assembly and use, for example during shipment and storage. FIG. 9 shows the autoinjector 10 after the needle guard 60 has been moved in the distal direction (normally by initiation of an injection). The distal movement of the needle guard 60 also pushes the lock activation sleeve 80 in the distal direction as well. Moving the lock activation sleeve 80 in the distal direction results in the pad 86 of the lock activation sleeve 80 engaging with the distal part 108 of the arm 104 of the lock component 101 and pushing the distal part 108 of the arm 104 towards the axis 20. The proximal part 106 of the arm 104 would pivot accordingly and move away from the axis 20, but the needle guard 60 stops this. As a result, the arm 104 is biased and is pushing (away from the axis) on the needle guard 60.

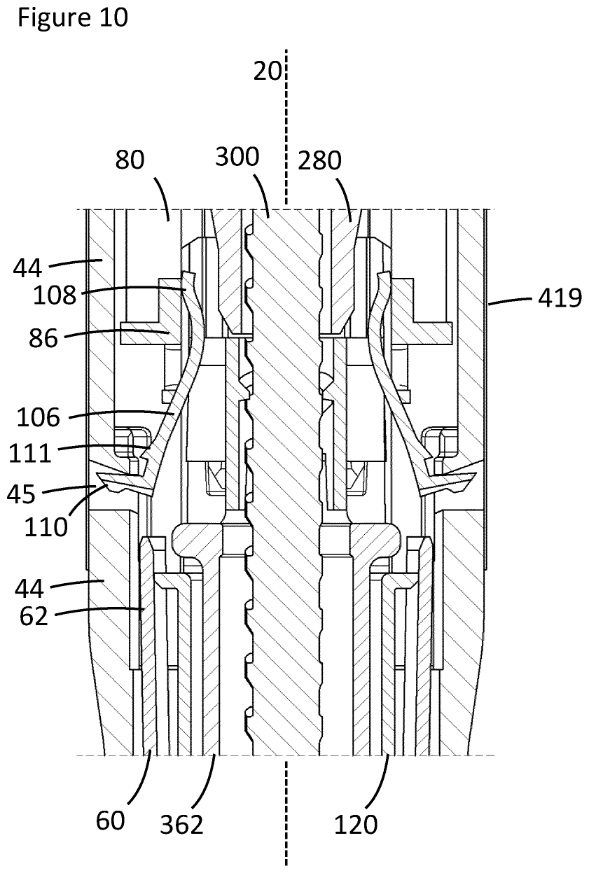

Once the needle guard is allowed to move in the distal direction again (typically once the injection is finished, though this could also be earlier in the case of a premature lifting of the needle guard from an injection site), the needle guard moves back in the proximal direction to a position as shown in FIG. 10 (in this case, the final position of the needle guard after injection is the same as the initial position before injection, though this is optional, and the final position after injection could be different from the initial position before injection; for example, the final position of the needle guard after injection could be further from the distal end of the housing than the initial position before injection (which might be beneficial in terms of needle safety), though the final position of the needle guard after injection could alternatively be closer to the distal end of the housing than the initial position before injection). The lock activation sleeve, however, does not move back to its original position and therefore the pad 86 is still pushing the distal part 108 of the arm 104 towards the axis 20. To release the resulting tension in the arm 104 as mentioned above, the proximal part 106 of the arm 104 moves away from the axis 20 once the needle guard 60 is no longer impeding it. The proximal part 106 of the arm 104 (or specifically in this example the protrusion 110 on the proximal part of the arm 104) ends up in (or adjacent to) the slit 45 in the housing 40. The arm may remain in tension after injection so that it holds the lock activation sleeve 80 in place by friction, although another feature or features elsewhere on the device could additionally or alternatively hold the lock activation sleeve in place.

FIG. 11 shows what happens if an attempt is made to push the needle guard 60 back in the distal direction after the lock created by the lock component 101 has been set. In this example, the needle guard 60 can move back in the distal direction a short way (a gap between the arm 104 and the needle guard 60 as shown in FIG. 10 is optional but preferable, as it allows for greater manufacturing tolerance during component manufacture and assembly), but is then stopped from moving further in the distal direction by the arm 104 of the lock component 101. The arm 104 is supported in the slit 45 (more generally a cut-out) by the protrusion 110. The slit 45 is shown extending through the housing, but this is not essential, and the slit could just be a recess in the inner surface of the housing (therefore only extending part of the way through the housing in the radial direction, and therefore not extending to the outer surface of the housing).

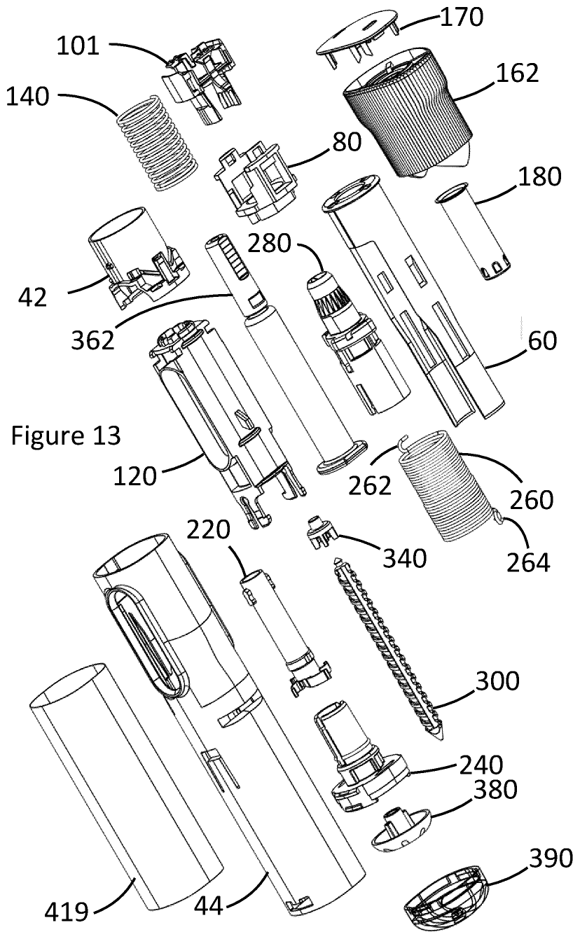

An example autoinjector 10 is shown in FIG. 12. The autoinjector 10 extends from a proximal end 14 to a distal end 16 along an axis 20, with an axial direction 22, a circumferential direction 24 and a radial direction 26 shown for reference. The autoinjector 10 comprises a housing 40 (which comprises a proximal housing (not visible) and a distal housing 44), a cap comprising a cap housing 162 and a cap insert 170. The housing comprises an optional window 46 and a neck 39. The neck 39 is optional, but is typically a ring around the housing that has a smaller external diameter than the rest of the housing (or at least a smaller external diameter than the housing immediately proximal and distal to the ring), and can help make it easier to grip the housing. The autoinjector comprises an optional label 419 extending around the housing 40, which can be used to provide information such as dosage and manufacturer to an end user, and/or which can help cover any features such as snap fits in the housing. The autoinjector also comprises a spinner cap 390. FIG. 13 shows an exploded perspective view of the autoinjector of FIG. 12, the autoinjector comprising a proximal housing 42, a distal housing 44, a needle guard 60, a lock activation sleeve 80, a lock component 101, a syringe holder 120, a needle guard spring 140, a cap housing 162, a cap insert 170, a rigid needle shield remover 180, a powerpack lock 220, a powerpack housing 240, a torsion spring 260, a driver 280, a plunger rod 300, a thrust bearing 340, a syringe 362, a spinner 380, a spinner cap 390, and a label 419.

A more detailed description of the functionality and components of the autoinjector of FIG. 12 will now be provided. For further details (and alternatives), reference is also made to published patent applications WO2022/078986 and WO2022/078987, both filed by SHL Medical AG, and both of which are hereby incorporated in their entirety by reference.

Starting roughly from the distal end of the device, the spinner cap 390 (FIG. 16) encloses the spinner 380 (FIG. 16). Although the spinner cap 390 is a separate component in this example (and is attached to the distal housing 44), the spinner cap 390 could alternatively be an integral part of a housing or an integral part of part of the housing, such as the distal housing 44. The spinner cap 390 is partially or fully see-through in this example, so that the spinner 380 can be seen by a user during medicament delivery. The spinner is optional; if there is no spinner, the spinner cap 390 is not needed (or at least does not need to be partially or fully see-through). Along with (or instead of) the spinner 380, other visual, audible or tactile forms of feedback could be additionally (or alternatively) provided, including the further protrusion 226 of the powerpack lock 220 as described below.

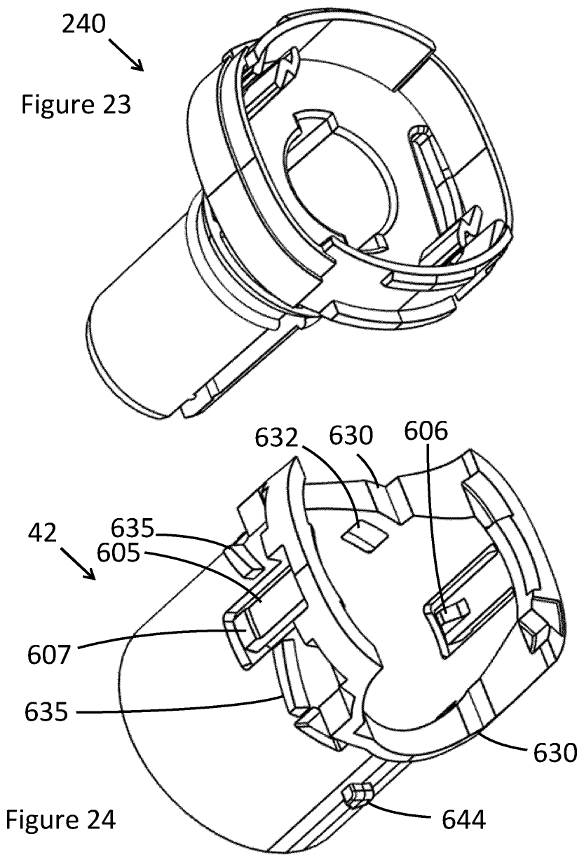

The spinner 380 is rotationally fixed relative to the powerpack lock 220 (FIG. 16), meaning that when the powerpack lock 220 rotates during medicament delivery, the spinner 380 also rotates. During medicament delivery, the powerpack lock 220 (FIG. 16) is rotationally fixed relative to the driver 280 (FIG. 15), and the driver 280 is rotated by the torsion spring 260. The torsion spring extends from a proximal end protrusion 262 of the torsion spring 260, which is attached to the driver 280, to a distal end protrusion 264 of the torsion spring 260, which is attached to the powerpack housing 240 (FIG. 23). The particular shapes of the torsion spring ends and the torsion spring attachment points can be varied and do not need to take the particular shape depicted. The powerpack housing 240 is rotationally fixed to the housing (in this case specifically to the distal housing 44). This means that the driver 280 is rotated relative to the housing by the torsion spring 260.

The plunger rod 300 (FIG. 16) is rotationally fixed relative to the driver 280, meaning that the plunger rod 300 rotates when the driver 280 rotates during medicament delivery. When the plunger rod 300 is rotated by the driver 280, a plunger rod screw thread 302 engages a lock component screw thread 322 (see also FIG. 113, for example), meaning that rotation of the plunger rod 300 relative to the lock component 101 results in the plunger rod 300 moving in the proximal direction relative to the housing. For completeness, it is noted that the lock component 101 is rotationally fixed relative to the housing (in this example the proximal housing 42 (FIG. 24) and the distal housing 44) so that the lock component 101 forces the plunger rod 300 in the proximal direction relative to the housing when the plunger rod 300 is rotated by the driver 280. For completeness, it is noted that the lock component is axially fixed relative to the syringe carrier (in the example given in FIG. 13), which in turn is axially fixed relative to the housing, although a direct attachment between the lock component and the housing to axially fix the lock component to the housing could alternatively be provided).

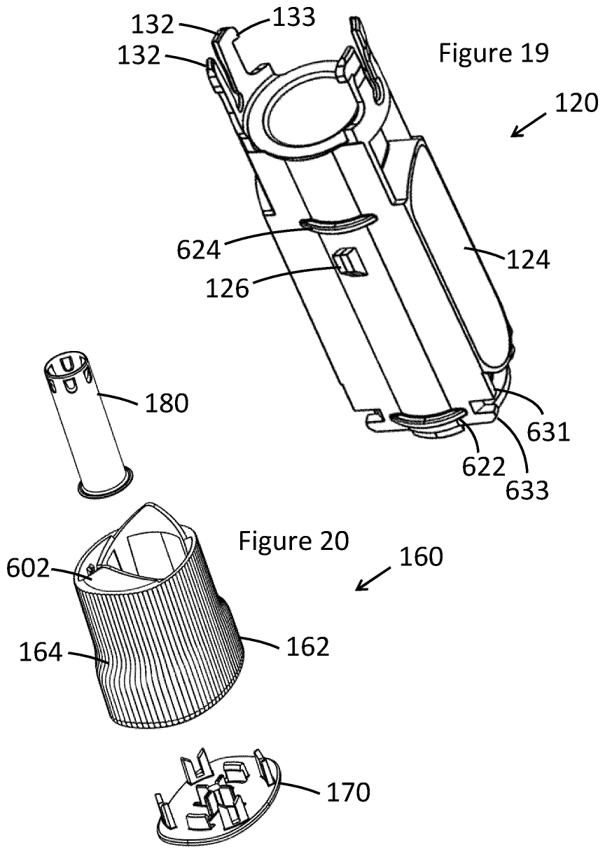

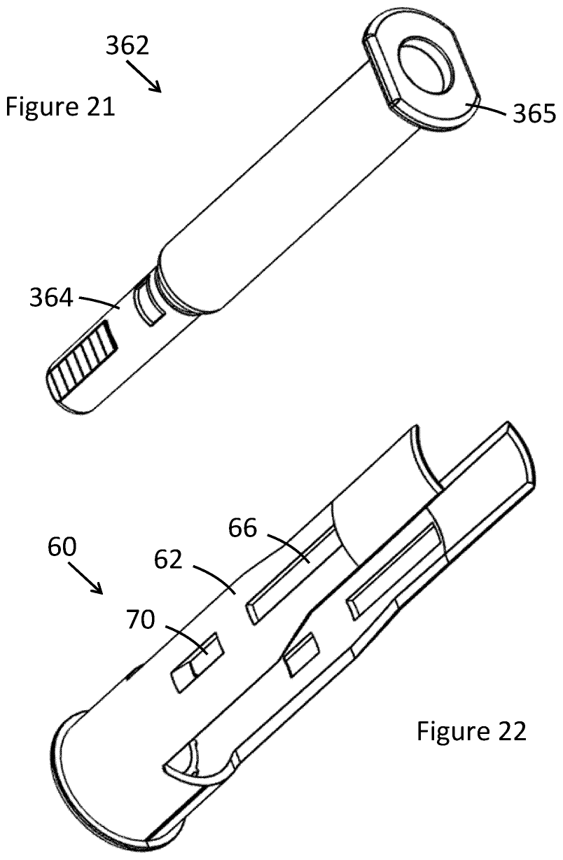

When the plunger rod 300 is rotated by the driver 280, thereby moving the plunger rod 300 in the proximal direction relative to the housing, the proximal end of the plunger rod 300 engages the thrust bearing 340 (FIGS. 17 and 18). The thrust bearing 340 is thereby pushed in the proximal direction relative to the housing, and the thrust bearing 340 in turn pushes the stopper 366 of the syringe 362 (FIG. 21) in the proximal direction relative to the housing, thereby expelling medicament from the syringe 362. The thrust bearing 340 is optional, but can be beneficially provided to reduce the friction between the plunger rod 300 and the stopper 366 of the syringe 362 (this can be beneficial to avoid rotation of the stopper (or even of the syringe) during medicament delivery; rotation of the stopper would cause extra friction and might potentially result in leakage of medicament past the stopper, and rotation of the syringe could cause issues with the needle 368 moving within a patient during medicament delivery). The syringe 362 is arranged inside a syringe holder 120 (FIG. 19), the syringe holder 120 being arranged inside the housing. Provision of a syringe holder 120 is optional though, and the syringe could instead be supported directly by the housing, for example (in other words, the housing would provide the functionality of a syringe housing within the same integral component, obviating the need for a separate syringe housing), or by another medicament delivery device component.

Turning to the proximal end of the autoinjector 10, an example cap 160 is provided (FIG. 20), with the cap 160 comprising three components, namely a cap housing 162, a cap insert 170 and a rigid needle shield remover 180, and with a needle guard 60 (FIG. 22) being arranged telescopically inside the housing. Optionally, the cap comprises a different number of components; for example, the cap housing 162 and the cap insert 170 could be a single integral part, and/or the rigid needle shield remover 180 could be integrated with the cap housing 162 or the cap housing 162 as a single integral part (particularly in examples where the rigid needle shield remover 180 is made of the same material as another component of the cap, for example a plastic). As a result, the cap can comprise one or more components.

The lock component 101 is described herein as part of a medicament delivery device sub-assembly with a lock activation sleeve 80, as part of a medicament delivery device sub-assembly with a housing, and as part of a medicament delivery device sub-assembly with a lock activation sleeve 80 and a housing. Another sub-assembly of a medicament delivery device comprises a housing, a cap and a syringe holder, and will now be described in more detail. This sub-assembly is shown in the context of the autoinjectors described herein, but could be implemented in various other types of autoinjector (or medicament delivery device more generally), including button-activated devices and devices that use other primary packages, such as cartridges rather than syringes, for example (and components such as the lock component 101, for example, are not essential features). Generally speaking, the sub-assembly comprises a housing, the housing being tubular and comprising a flexible arm, the flexible arm comprising a protrusion extending towards the axis and a surface facing away from the axis; a cap, the cap being removably attached to a proximal end of the housing, the cap comprising a surface facing towards the axis; a syringe holder attached to the housing, the syringe holder comprising a protrusion extending away from the axis; a medicament delivery member guard arranged telescopically at least partially inside the housing, the medicament delivery member guard comprising a cut-out; wherein the surface of the cap is adjacent to the surface of the housing to restrict movement of the flexible arm away from the axis when the cap is attached to the housing; wherein the protrusion of the flexible arm is in the cut-out of the medicament delivery member guard; and wherein the protrusion of the syringe holder is adjacent to the protrusion of the flexible arm of the housing, so that, when the cap is attached to the housing, the protrusion of the flexible arm of the housing and the protrusion of the syringe holder together restrict distal movement of the medicament delivery member guard relative to the housing.

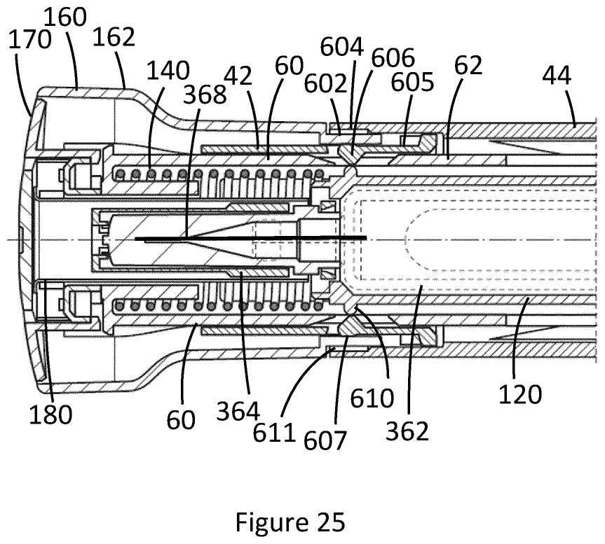

This sub-assembly will now be put into context in the example of FIG. 25, which shows the cap 160 relative to various other components of the autoinjector 10, namely the proximal housing 42, the distal housing 44, the needle guard 60, the syringe holder 120, the needle guard spring 140 and the syringe 362. The interplay between these components will now be described, with particular focus on the proximal housing 42, the syringe holder 120 and the cap housing 162 of the cap 160.

The cap housing 162 comprises a distally extending protrusion 602. As shown in FIG. 25, the distally extending protrusion 602 is arranged between two parts of the housing, with an outer part of the housing being radially outside (further from the axis than) the distally extending protrusion 602 and an inner part of the housing being radially inside (closer to the axis than) the distally extending protrusion 602. The outer part is an inwardly facing surface 604 of the distal housing 44; the inwardly facing surface 604 can be tapered, with a distal end of the inwardly facing surface 604 being closer to the axis than a proximal end of the internally facing surface; the tapering is optional but can help with assembly. The inwardly facing surface 604 of the distal housing 44 can help restrict outward flexing (away from the axis) of the distally extending protrusion 602 of the cap housing 162. The provision of an inwardly facing surface 604 of the distal housing 44 is optional, as if the distally extending protrusion 602 of the cap housing 162 is rigid, the support of the inwardly facing surface 604 of the distal housing 44 would be optional. For further reference, a close-up of a similar embodiment to that in FIG. 25 is provided in FIG. 46 (in FIG. 46, the syringe holder 120 is not shown).

In FIG. 25, an optional gap 611 (or housing recess) can be seen between the distally extending protrusion 602 of the cap housing 162 and the internally inwardly facing surface 604 of the distal housing 44. This gap can provide space for the flexible arm 605 of the proximal housing 42 to move out of the way of the needle guard 60 once the cap is removed from the device. optionally, the gap is narrower in the circumferential direction than the distally extending protrusion 602 of the cap housing 162. This would mean that the distally extending protrusion 602 of the cap housing 162 cannot enter the gap between the distally extending protrusion 602 of the cap housing 162 and the internally inwardly facing surface 604 of the distal housing 44. The flexible arm 605 of the proximal housing 42 could then be provided with a circumferential width that is narrower than (or the same width as) the circumferential width of the gap (or at least, a outwardly extending protrusion 607 of the flexible arm 605 is narrower than the circumferential width of the gap), thereby allowing (part of) the flexible arm 605 to enter the gap.

The inner part of the housing is a flexible arm 605 of the proximal housing 42. The flexible arm 605 is attached to the rest of the proximal housing 42 at the distal end of the flexible arm 605, although the flexible arm 605 could alternatively be attached to the rest of the proximal housing 42 at the proximal end of the flexible arm 605, or could even extend in a direction other than the axial direction. The flexible arm 605 comprises an inwardly extending protrusion 606 and an outwardly extending protrusion 607, with the inwardly extending protrusion 606 being arranged to abut a corresponding outwardly extending protrusion 610 of the syringe holder 120 and the outwardly extending protrusion 607 being arranged to abut the distally extending protrusion 602 of the cap housing 162. The flexible arm 605 of the proximal housing 42 extends into a cut-out 70 of the needle guard arm 62 (FIG. 22). As a result of this combination of features, the needle guard 60 is blocked from moving in the distal direction (relative to the housing) before the cap is removed, as the housing, cap, and syringe holder are all in contact, meaning that the distal movement of the needle guard 60 is limited. Although the outwardly extending protrusion 610 of the syringe holder 120, for example, is optional, most of the components in the autoinjector 10 will typically be somewhat flexible, meaning that without the outwardly extending protrusion 610 of the syringe holder 120, the needle guard 60 (and particularly the needle guard arm 62) will generally be somewhat flexible, potentially allowing the needle guard arm 62 to deform slightly to pass the flexible arm 605 of the proximal housing 42 if the outwardly extending protrusion 610 of the syringe holder 120 is not provided. As such, the applicant has appreciated that the provision of this particular combination of features of the housing, cap and syringe holder can be beneficial. As the cap is removable from the medicament delivery device: as well as being relevant to the level of drop protection provided in a complete medicament delivery device, the precise dimensions of these features could also be tweaked to adjust cap removal force, as the cap can be held in place (primarily prior to device use, but also after recapping after device use if recapping is performed) by having the distally extending protrusion 602 of the cap housing 162 gripped between the inwardly facing surface 604 of the distal housing 44 and the outwardly extending protrusion 607 of the flexible arm 605.

The protrusion 606 and the protrusion 607 of the proximal housing 42 are optional, and their functionality could more generally be provided by surfaces of the flexible arm 605 of the proximal housing 42 (an inwardly facing surface instead of the protrusion 606, and an outwardly facing surface instead of the protrusion 607) rather than by protrusions.

Although the inwardly extending protrusion 606 of the flexible arm 605 and the outwardly extending protrusion 610 of the syringe holder 120 optionally touch one another, this is not essential. Alternatively, for example, the cut-out 70 is replaced by a recess. The recess is preferably in the outer surface of the needle guard arm 62; the recess could alternatively be in the inner surface of the needle guard arm 62, with the outwardly extending protrusion 610 of the syringe holder 120 arranged in the recess, though this would require bending of the needle guard arm 62 (or bending of the syringe holder 120) for the needle guard arm 62 to pass the outwardly extending protrusion 610 of the syringe holder 120 and activate medicament delivery, which is possible but may be suboptimal depending on the flexibility of the needle guard arm 62, as friction between the outwardly extending protrusion 610 of the syringe holder 120 and the needle guard arm 62 may make it hard for a user to activate the device). Optionally, two recesses (one in the inner surface of the needle guard arm 62 and one in the outer surface of the needle guard arm 62) could be provided, with one or both of the corresponding protrusions 606, 610 in the corresponding two recesses. The depth of the recess (or the combined depth of the recesses where two recesses are provided) would be greater than the combined spaces between the distally extending protrusion 602 of the cap housing 162 and the flexible arm 605 of the proximal housing 42, and between flexible arm 605 of the proximal housing 42 and the outwardly extending protrusion 610 of the syringe holder 120. This can restrict movement of the needle guard arm 62 in the distal direction relative to the housing (prior to cap removal). This can provide a reliable pre-activation lock of the needle guard 60. In an another approach, the outwardly extending protrusion 610 of the syringe holder 120 is optional, and if needed, the flexible arm 605 of the proximal housing 42 (particularly the inwardly extending protrusion 606 of the flexible arm 605 of the proximal housing 42) could extend further towards the axis to replace the outwardly extending protrusion 610 of the syringe holder 120, which would typically mean that the inwardly facing surface of the arms 62 of the needle guard 60 is further from the axis than the innermost part of the flexible arm 605 of the proximal housing 42 (particularly than the innermost part of the outwardly extending protrusion 607 of the flexible arm 605 of the proximal housing 42).

Another factor that can further help with providing a secure pre-activation lock of the needle guard 60 is control over ovalisation of the syringe holder 120 and/or of the housing (specifically the proximal housing 42 and/or the distal housing 44). Optionally, the gap between the syringe holder and the housing (specifically the distal housing 44 in the depicted examples) is minimised or removed entirely to minimise the possibility for ovalisation of the syringe holder. This is to help ensure that the drop activation lock described herein (particularly with reference to FIG. 25) cannot be overridden by deformation of components. The syringe holder and the housing can be adjacent to one another at some points around the circumference of the syringe holder, but typically not around the entire circumference of the syringe holder (because the needle guard is in the way, for example). One area where the syringe holder and the housing can be adjacent to one another is with the respective windows 46, 124. A form fit between the window 46 of the housing 40 and the window 124 of the syringe holder 120 can help minimise ovalisation of the syringe holder 120. Other factors, such as structural features such as ribs, can directly (i.e. structural features of the syringe holder) or indirectly (i.e. structural features of other components, such as the distal housing 44) control, limit and/or stop ovalisation of the syringe holder.

In the examples described above, the proximal housing 42 has a flexible arm 605 and the needle guard 60 has a cut-out and the cap is the added/removed member in the chain of blocking components (from outermost to innermost housing (optional), cap, syringe holder, needle guard, syringe housing), with the removal of the cap giving space for the flexing of the flexible arm 605 of the proximal housing 42 to occur. These roles can be changed around (and the outwardly extending protrusion 610 of the syringe holder 120, plus optional ovalisation prevention, can still be included). For example, the needle cover can have a rib and the front insert can have a recess, with the rib in the recess. The syringe holder and the proximal housing 42 a single integral component. In general, an alternative needle guard lock could also be provided, or the needle guard lock could be left out altogether (for example if the lock provided by the lock component 101 is considered to be sufficient without the additional lock on the needle guard).

Various details and further alternatives for components of the autoinjector described above will now be outlined. An example syringe holder 120 (or syringe carrier, or more generally a medicament delivery container holder) is shown in FIG. 19. The syringe holder comprises an optional window 124 (which is typically aligned with the window of the housing in an assembled medicament delivery device). The syringe holder can be attached in various ways to various components (primarily to components that do not move axially relative to the housing during device use) such as the housing and/or the lock component 101. In the main example described herein, the interaction of a snap-fit arm 132 of the syringe holder 120 (FIG. 19) and a snap-fit hole 336 of the lock component 101 (FIG. 1) (in this example, four snap-fit arms 132, each comprising a circumferentially-extending protrusion 133 and four corresponding snap-fit holes 336) is provided to attach the syringe holder to the lock component, although other attachment mechanisms could alternatively or additionally be provided. The syringe holder also comprises a protrusion 126 which extends outwardly from an outer surface of the syringe holder. The protrusion 126 aligns with a slit 66 of the needle guard arm 62 (FIG. 22), thereby restricting rotational movement of the needle guard 60 relative to the syringe holder—this is not essential, but can be beneficial to guide the movement of the needle guard 60. Optionally, the proximally facing edge of the protrusion 126 of the syringe holder 120 is chamfered (e.g. FIG. 19), which can help the needle guard 60 to pass the protrusion 126 of the syringe holder 120 during assembly.

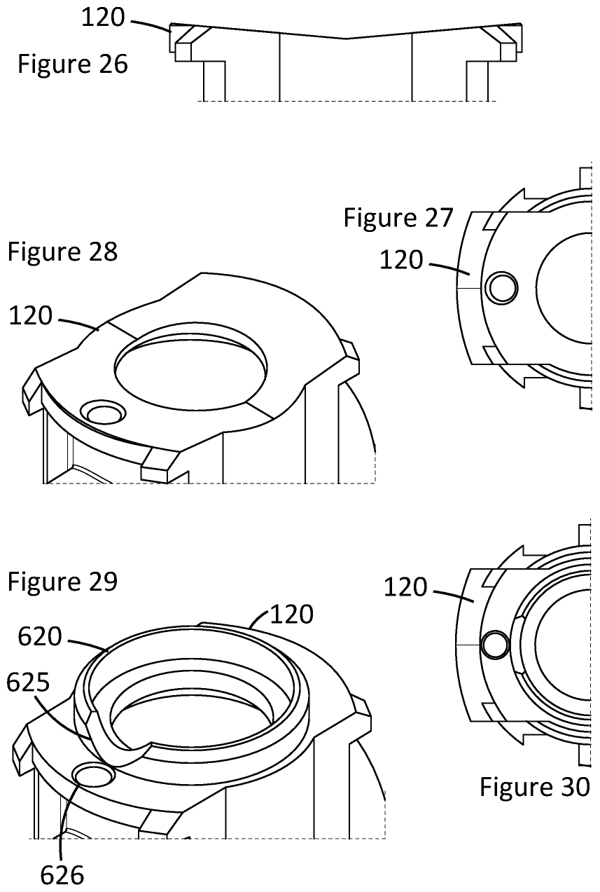

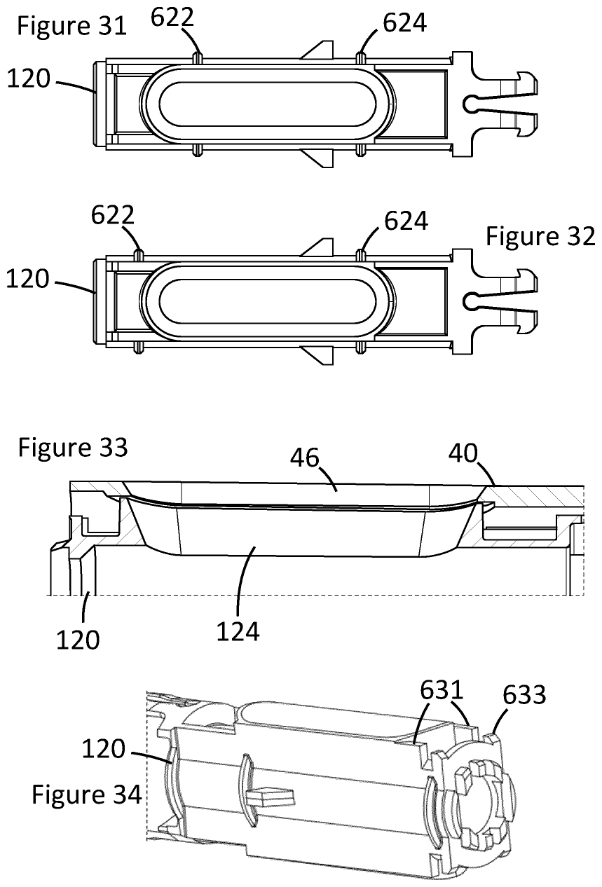

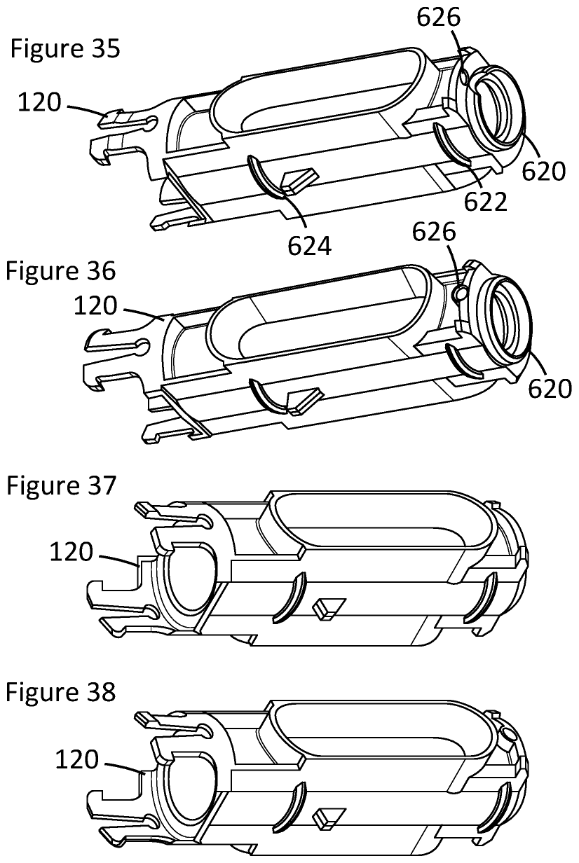

FIGS. 26 to 44 will now be used to describe various examples of a syringe holder and to show some of the possible variations in design of the syringe holder. FIGS. 26 to 28 show views of the proximal end of an example syringe holder without a support ring, and FIGS. 29 and 30 show views of an example syringe holder with a support ring 620. The support ring 620 is optional, and can support the distal end of the needle guard spring 140. As shown in FIG. 26, the proximal surface of the syringe holder can be non-planar (in this case V-shaped), or can be planar (as in FIG. 29, where the support ring 620 extends in the proximal direction from the proximal surface of the syringe holder). The example in FIG. 29 shows a support ring 620 with a notch 625; the notch is optional and is provided to allow space for the injection moulding gate 626. An alternative without a notch 625 is shown in FIG. 36, where the location of the gate 626 is different.

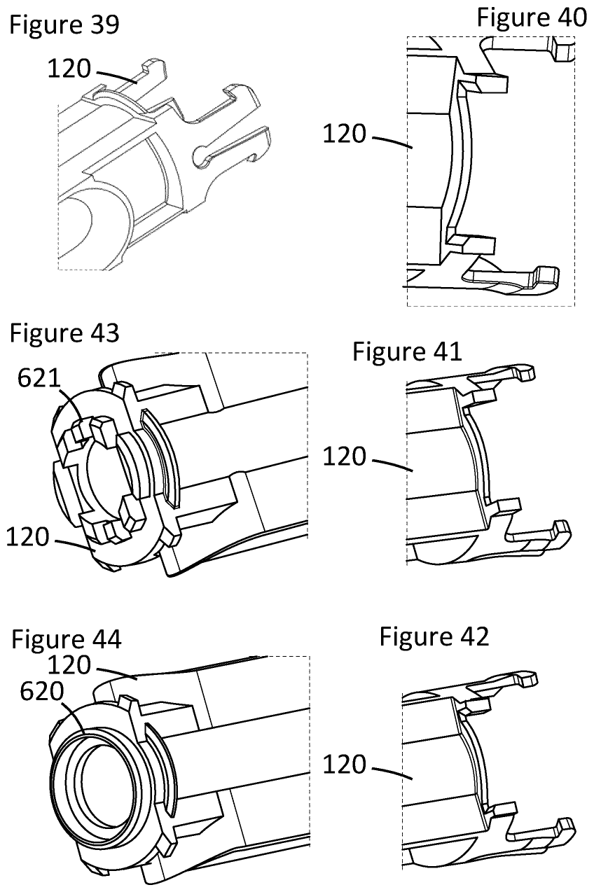

Optionally, the syringe holder comprises one or more support ribs, such as a proximal support rib 622 and/or a distal support rib 624 (see FIGS. 19, 31, 32 and 35 to 38, for example). These support ribs 622, 624 can help provide rigidity and/or strength, for example. The positions of the support ribs can be varied depending on required strength profiles, required rigidity profiles, and/or the shape of other medicament delivery device components, for example. Optionally, the proximal support rib 622 is the outwardly extending protrusion 610 (thereby potentially fulfilling two functions). The window of the syringe holder would typically be aligned with the window of the housing as shown in FIG. 33. FIGS. 39 to 42 show various minor adjustments that could be made to the distal end of the syringe holder, for example to optimise moldability, to accommodate the shape of other medicament delivery device components, to achieve desired rigidity profiles, and/or to achieve desired strength profiles. FIG. 43 shows an alternative syringe holder design in which a broken support ring 621 is provided (i.e. a ring made up of a plurality of spaced apart protrusions, see also FIG. 34 for another example), rather than the continuous support ring 620 shown in FIGS. 29, 35 to 38, and 44 (i.e. a ring that extends 360 degrees around the axis). A broken ring can be beneficial in terms of tooling for medicament delivery device assembly.

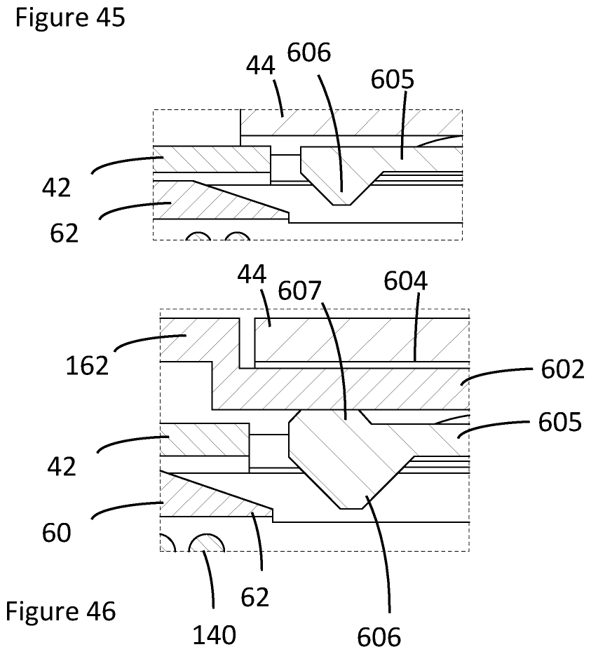

FIG. 45 shows a close-up of an alternative, showing in particular parts of the cap housing 162, the housing and the needle guard (specifically the needle guard arm 62), in which a distally extending protrusion of the cap housing is not provided.

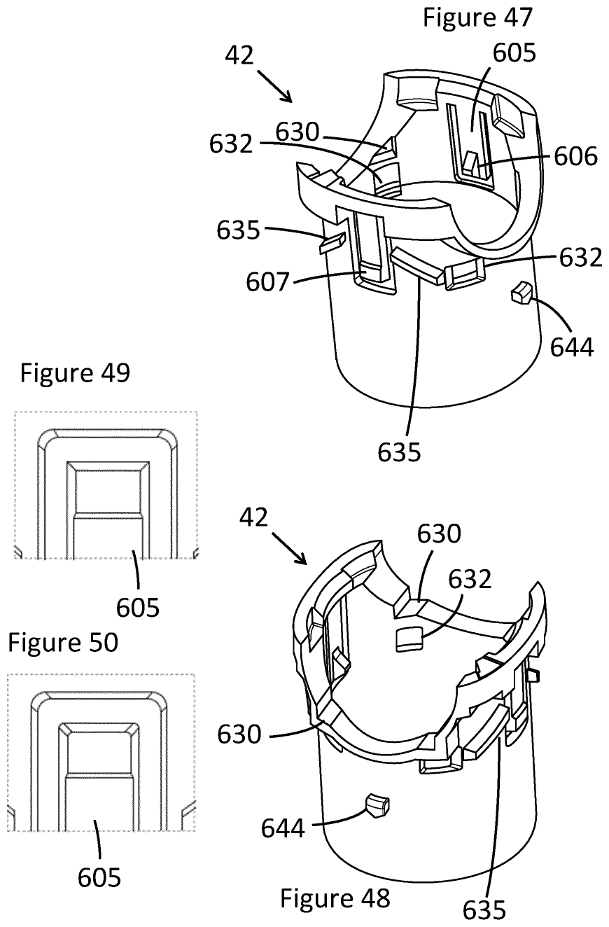

FIG. 48 shows a different view of the proximal housing 42 of FIG. 24, and FIGS. 47 and 49 to 52 show alternatives in the design of the proximal housing 42. As shown in FIGS. 24 and 48, the proximal housing 42 comprises the flexible arm 605 as described elsewhere, which is arranged in a cut-out in the proximal housing. Several optional features of the proximal housing 42 are designed to engage with corresponding features of the syringe holder 120. In particular, the proximal housing 42 comprises a notch 630 and a cut-out 632, or more specifically four notches 630 and four cut-outs 632. Each notch 630 is arranged on an inwardly facing surface of the housing, and is axially aligned with a corresponding cut-out 632, with the cut-out being arranged closer to the proximal end of the proximal housing 42 than the notch. In an assembled medicament delivery device, the notches 630 engage corresponding ribs 631 of the syringe holder 120 (see FIGS. 19 and 34, for example). The notches 630 and ribs 631 are optional, but can help correctly arrange the proximal housing 42 and the syringe holder 120 relative to one another. In an assembled medicament delivery device, the cut-outs 632 are one half of a snap-fit, with the other half of the snap-fit being protrusions 633 on the syringe holder 120 (see FIGS. 19 and 34, for example); this can attach the proximal housing 42 to the syringe holder 120, and can also rotationally lock the proximal housing 42 to the syringe holder 120.

Some interactions between the cap 160 and the proximal housing 42 are already described above, particularly with respect to the distally extending protrusion 602 of the cap housing 162 and the flexible arm 605 of the proximal housing 42 (see FIG. 25 in particular). In addition, optional ribs 635 of the proximal housing 42 are arranged to engage the distally extending protrusion 602 of the cap housing 162. More specifically, the ribs 635 protrude from an outer surface of the proximal housing 42, and each of the ribs 635 comprise a proximally facing surface which is angled to engage a corresponding distally facing surface of a distally extending protrusion 602 of the cap housing 162. In this example, the proximally facing surfaces of the ribs are angled away from the axis, though this is optional. The ribs are arranged in two pairs. In each pair of ribs, the two ribs are arranged with one rib on each side of the flexible arm 605 of the proximal housing 42. Each rib extends at an angle relative to the circumferential direction around the axis, with the ribs effectively in a V shape (but with the base of the V removed, to allow space for the flexible arm 605). The V shape is mirrored by the distally extending protrusion 602 of the cap housing 162, with the distally facing surfaces of the distally extending protrusion 602 also defining a V shape. In an assembled medicament delivery device, the V shape of the distally extending protrusion 602 of the cap housing 162 sits inside the V shape formed by the pair of ribs 635. As a result, if the cap 160 is then rotated relative to the proximal housing 42, the cap is forced in the proximal direction relative to the proximal housing 42. This feature does not, however, require a rotational removal of the cap, and the cap could also be removed axially. The ribs 635 and the protrusions 602 are optionally in contact (in an assembled medicament delivery device), but not necessarily, and a small gap between the ribs 635 and the protrusions 602 could help reduce the force required to start rotation of the cap relative to the proximal housing. Although the protrusions 602 shown herein extend beyond the main body of the cap housing 162 and have a triangular shape, it is noted that the protrusion 602 could simply be a distal part of the cap. Optionally, the cap housing 162 comprises inwardly extending ribs 646 that further extend the V shape of the cap housing, thereby further supporting the ribs 635 of the proximal housing 42 to help further guide the cap relative to the proximal housing during cap removal.

The proximal housing 42 comprises an outwardly extending protrusion 644, with the cap housing 162 comprising a corresponding inwardly extending protrusion 645 (FIG. 58, for example). These protrusions 644, 645 are optional, but can help hold the cap in place relative to the proximal housing in an assembled medicament delivery device. The particular shapes of the protrusions 644, 645 can also be altered, for example to alter the force required to remove the cap. FIGS. 49 to 51 show options for minor alterations to the shape of the flexible arm 605 of the proximal housing 42, which can have rounded or sharp corners at the proximal end of the flexible arm 605.

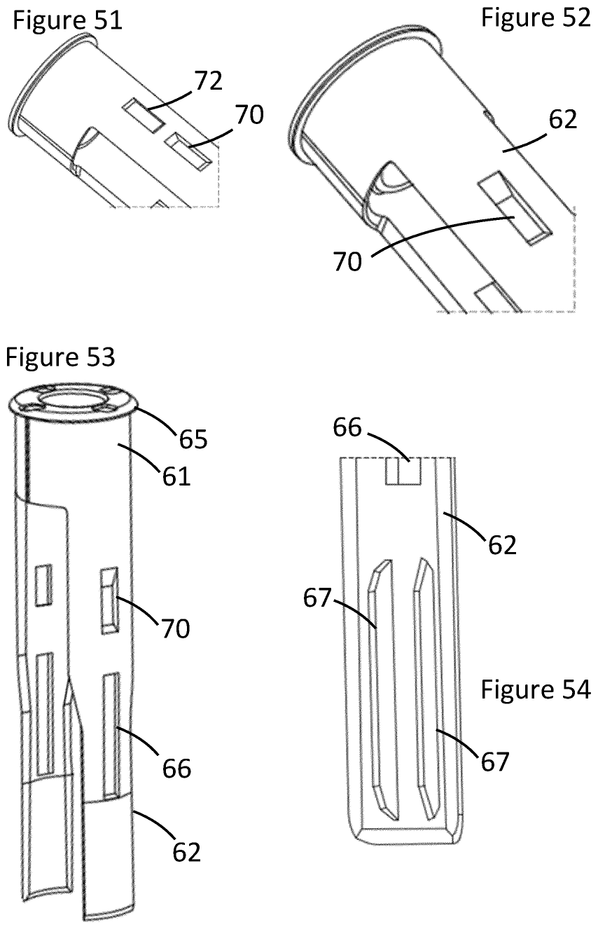

FIGS. 51 to 54 will now be used to describe the needle guard 60 in more detail. FIG. 51 shows an alternative design in which a recess 72 is arranged in the outer surface of the needle guard 60 (specifically of the needle guard arm 62), the recess 72 being closer to the proximal end than the cut-out 70 and axially in line with the cut-out 70. The recess 72 is optional, but can be useful to decrease friction in some embodiments. FIGS. 52 and 53 show different views of the needle guard 60 of FIG. 22. As shown in FIG. 52 in particular, the needle guard 60 comprises a base 61, which in this example is cylindrical, two arms 62 extending in the distal direction from the base 61, and a flange 65 attached at the proximal end of the base 61. The flange 65 is optional, and the particular shape of the flange can be varied depending on various factors including the patient group for a particular drug; for example, a larger flange could be provided for a drug that is likely to be used by obese patients. Four optional indents are visible in the flange (FIG. 53). The cut-out 70 optionally has chamfered edges at the proximal and distal ends of the cut-out 70. The chamfered edge at the proximal end is optionally angled less than the chamfered edge at the distal end (that is, the face of the chamfered edge at the proximal end extends in a direction that is closer to parallel to the axis than the face of the chamfered edge at the distal end). FIG. 54 shows ribs 67 that extend from the inside face of the needle guard arm 62 and extend parallel to the axis; these ribs 67 are optional, but can be included to improve rigidity, for example. Two different options for the rib shape are shown for illustrative purposes on the same needle guard arm 62 in FIG. 54, though in a practical design the two ribs would typically be the same.

In the depicted examples, the needle guard 60 comprises a base 61 of the needle guard 60, which is cylindrical, and two needle guard arms 62, though the shape of the base and of the arms (and the number of arms) can be varied depending, for example, on the shape of other medicament delivery device components.

FIGS. 55 to 64 will now be used to describe the cap 160 in more detail. FIGS. 55 to 56 show an example of the internal structure of the cap housing 162. FIGS. 57 to 58 show another example of the internal structure of the cap housing 162; this design is preferable to the design in FIGS. 55 to 56 from a moulding perspective, due to the different angles and thicknesses of several of the internal support ribs. A protrusion 645 of the cap housing 162 is also visible extending inwardly from an inner surface of the cap housing 162; the protrusion 645 interacts with the protrusion 644 of the proximal housing 42 as described above. A flange 650 of the cap housing 162 comprises a proximally facing surface that is arranged to abut a distally facing surface of the flange 182 of the rigid needle shield remover 180, as shown in FIGS. 59 and 60. Optionally, the portion of the proximally facing surface of the flange 650 of the cap housing 162 is chamfered, as shown in FIG. 60 (rather than a right-angled edge, as shown in FIG. 59); this can help during assembly, for example by allowing greater placement tolerance when inserting the rigid needle shield remover 180 into the cap housing 162.

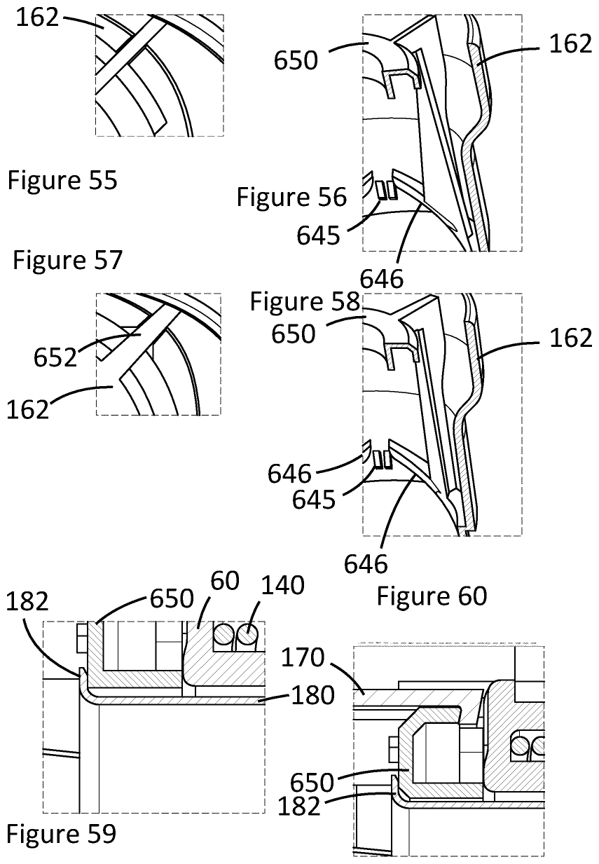

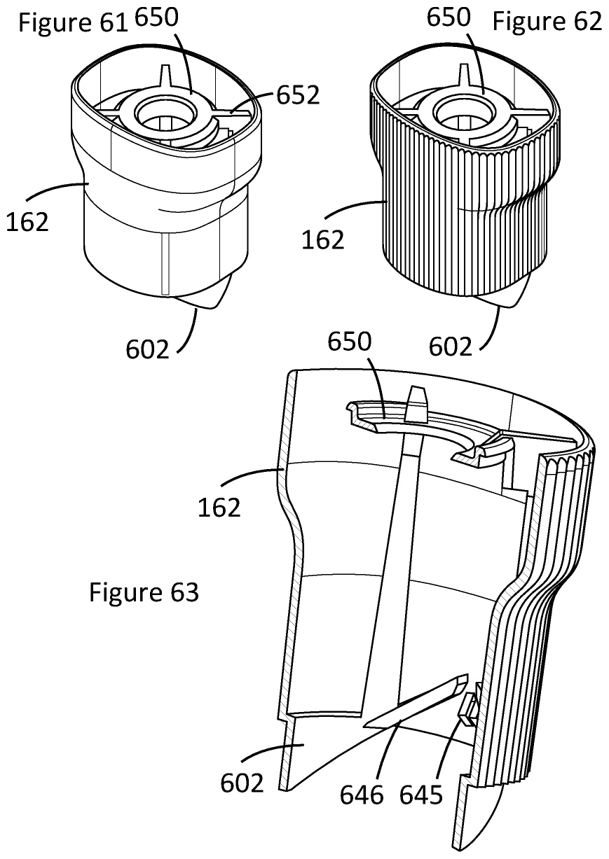

FIG. 61 shows the cap housing 162 of FIG. 20 without a grip 164; in general, the grip is optional. FIG. 62 shows a further view of the cap housing 162 of FIG. 20, where an outer surface of the cap housing 162 comprises an optional grip 164. In this example, the grip 164 is a plurality of longitudinally extending ribs spaced around the outer surface of the cap housing, although other types of grip could be additionally or alternatively be provided, such as circular protrusions or a separate grip that is attached to the outer surface of the cap housing 162.

FIG. 63 shows another embodiment of a cap housing 162, showing an example of various minor structural changes that could be made to features such as the flange 650, for example to optimise the shape for moulding and to adjust features of the cap housing to account for structural changes in other medicament delivery device components. FIG. 64 shows a partially see-through perspective view of part of the cap housing 162 of FIG. 63. As shown in FIG. 60, for example, a distally facing surface of the flange 650 of the cap housing 162 engages a first arm 660 of the cap insert 170. In an alternative approach, the cap housing 162 and the cap insert 170 could be a single integral part. In another alternative approach, the cap could be a single integral part. The rigid needle shield remover 180 (which can be generalised to rigid medicament delivery member shield remover for alternatives in which a medicament delivery member other than a needle is used) can be made out of plastic or metal, for example. In some alternative approaches, various features of the cap, such as the flange 650 and rib 652 of the cap housing 162 and the first arms 660 and second arms 662 of the cap insert 170, are optional, particularly if two or more of the cap housing 162, the cap insert 170 and the rigid needle shield remover 180 are a single integral part, or if another approach to attaching the rigid needle shield remover 180 to the rest of the cap is used, for example welding or gluing.

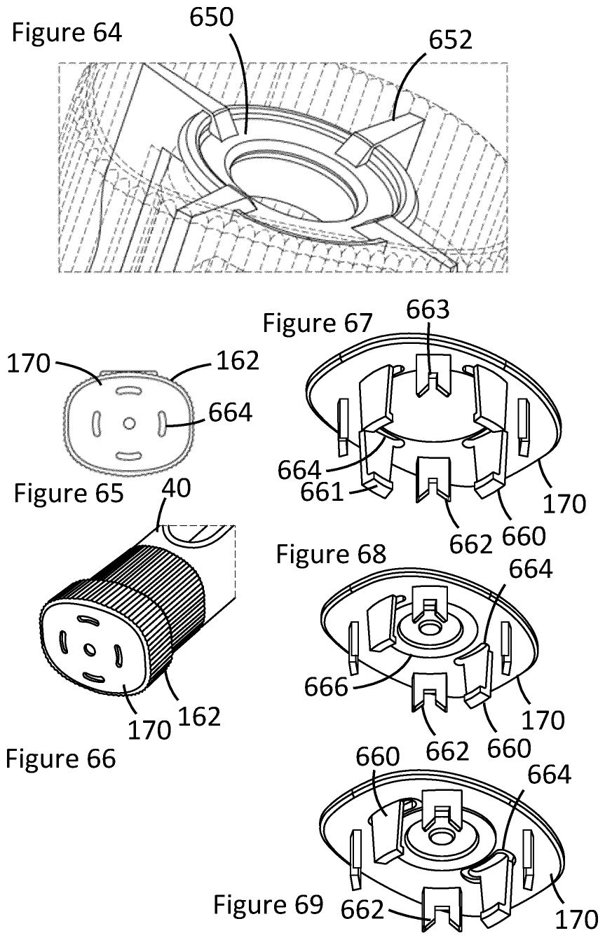

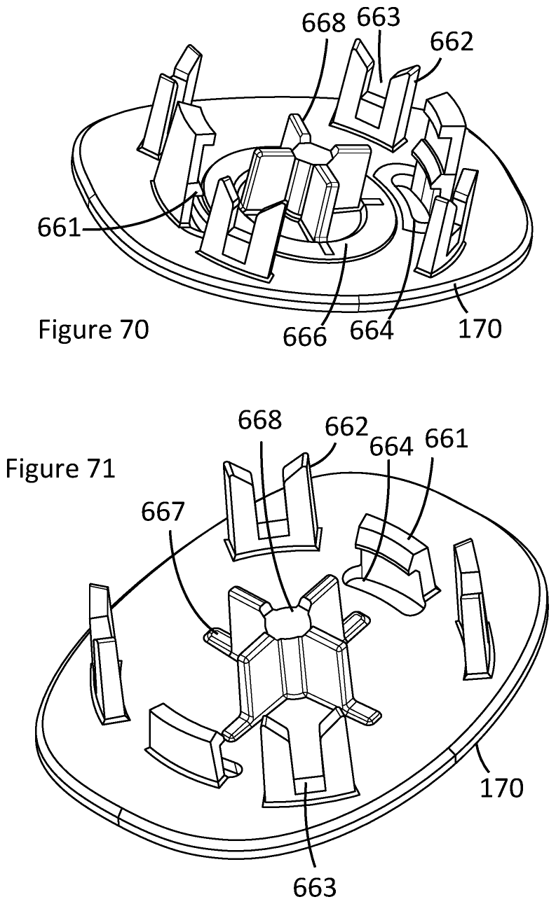

FIGS. 65 to 71 will now be used to describe the cap insert 170 in more detail. FIGS. 65 to 67 show an example cap insert 170, with FIGS. 65 and 66 showing the cap insert 170 in the context of a housing 40 and a cap housing 162. FIGS. 68 to 71 show four more examples of cap housings 162 with somewhat different dimensions for various features including those above; these modifications can be adjusted in line with modifications to other medicament delivery device components or to modify cap rigidity, for example.

The cap insert 170 comprises first arms 660 that extend in the axial direction (e.g. FIG. 67), with each of the first arms comprising an inwardly facing protrusion 661, the inwardly facing protrusion comprising a proximally facing surface that engages with a distally facing surface of the cap housing 162 (with a distally facing surface of the flange 650 in this example), thereby creating a snap-fit between the cap insert 170 and the cap housing 162. The cap insert 170 comprises second arms 662 that extend in the axial direction (e.g. FIG. 67); each of the second arms comprises a notch 663 in the distal end of the arm 662, which engage a rib 652 of the cap housing 162 (see e.g. FIG. 64); the rib 652 extends in the radial direction. The second arms 662 can thereby restrict or stop rotation of the cap housing 162 relative to the cap insert 170. Overall, the combination of the first arms 660 and second arms 662 of the cap insert 170 and the flange 650 and rib 652 of the cap housing 162 attach the cap housing 162 to the cap insert 170. Optionally, the cap insert 170 also comprises air holes 664 (e.g. FIG. 69) as a safety feature against choking in the event that the cap is accidentally swallowed. The holes 664 may also be helpful for mouldability of the cap insert 170. Optionally, a ring-shaped protrusion 666 of the cap housing 162 is provided (e.g. FIGS. 68 to 70); this can provide a planar surface to abut the proximal end of the rigid needle shield remover 180 and thereby support the rigid needle shield remover 180 between the cap housing 162 (or more specifically the flange 650 of the cap housing 162) and the cap insert 170. Alternatively, instead of a ring-shaped protrusion of the cap housing, a plurality of support ribs 667 (e.g. FIG. 71) can be provided for the same purpose. Optionally, a protrusion 668 of the cap insert 170 extends along the axis; this protrusion extends inside the rigid needle shield remover 180 and can help support correct positioning of the rigid needle shield remover 180 within an assembled cap. Optionally, as can be seen in FIGS. 61 and 62, the cap (specifically an outer surface of the cap such as an outer surface of a cap housing 162) is non-circular when viewed along the axis. This can help a user grip the cap more strongly and can therefore help a user impart more force to help remove the cap from the device.

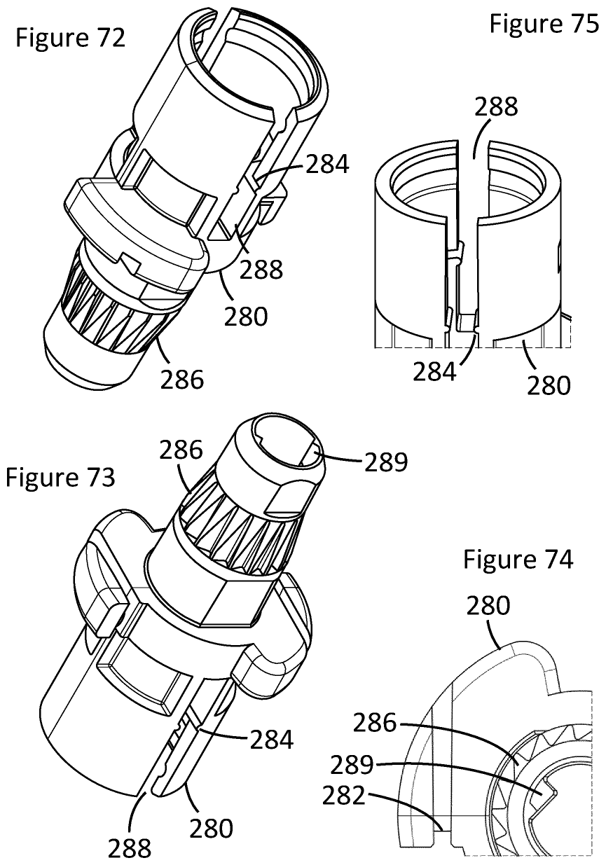

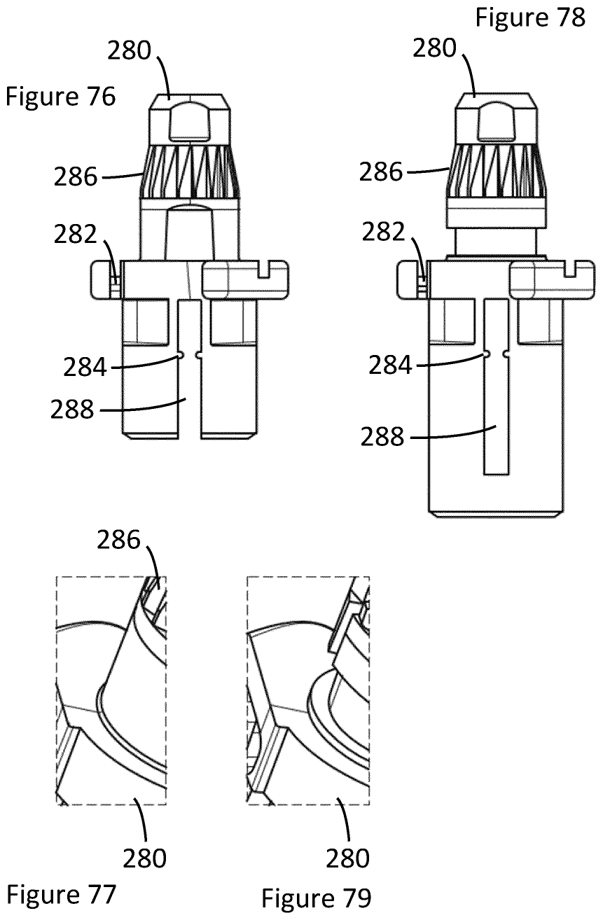

FIGS. 72 to 79 will now be used to describe the driver 280 in more detail using several different example drivers. The driver 280 comprises a circumferentially facing ledge 282, which engages a proximal end protrusion 262 of the torsion spring 260. The driver 280 comprises an optional protrusion 284, which engages the protrusion 224 of the powerpack lock 220 (e.g. FIG. 16), providing a restriction on the axial movement of the powerpack lock 220 relative to the driver 280. The driver 280 comprises a driver slot 288; the protrusion 224 of the powerpack lock 220 is arranged in the driver slot 288, thereby restricting rotational movement of the driver 280 relative to the powerpack lock 220. Optionally, as shown in the examples in FIGS. 72 and 75 for example, the driver slot 288 extends from the distal end of the driver 280. Alternatively, the driver slot is spaced apart from the distal end of the driver 280, as shown in the examples in FIGS. 15 and 78; this can be advantageous as it can provide a more rigid driver structure. The driver 280 comprises driver teeth 286, which engage the corresponding lock component teeth 326 to lock the driver 280 to the lock component 101 prior to activation of the medicament delivery device. The driver 280 comprises an inwardly extending rib 289 (e.g. FIGS. 73 and 74) comprising a circumferentially facing surface. The circumferentially facing surface engages a corresponding circumferentially facing surface 305 of the plunger rod 300 (see FIG. 16; in this example, the circumferentially facing surface 305 is adjacent to a flattened side 304 of the plunger rod 300). In general, the plunger rod is non-circular when viewed in cross-section relative to the axis, with the inner surface of the driver being adjusted to the shape of the plunger rod, so that the driver and the plunger rod are rotationally bound to one another (so that rotation of the driver by the torsion spring is transferred into rotation of the plunger rod).

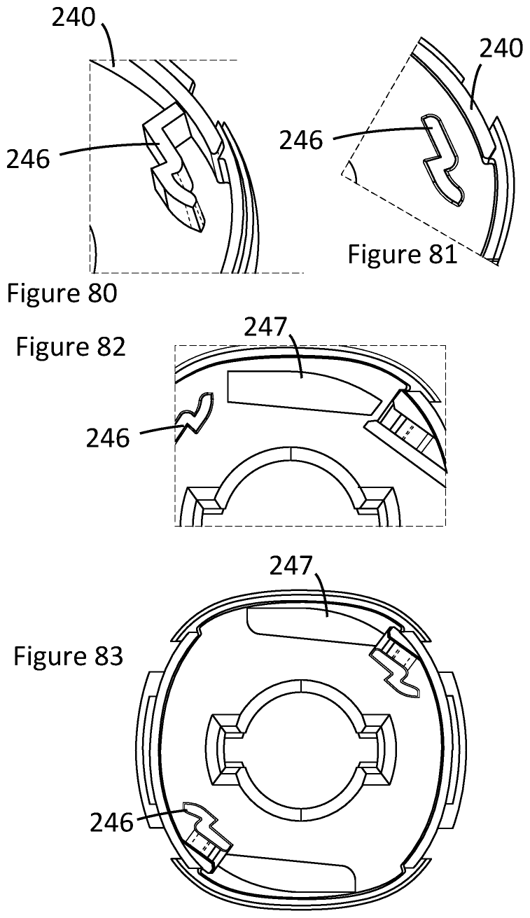

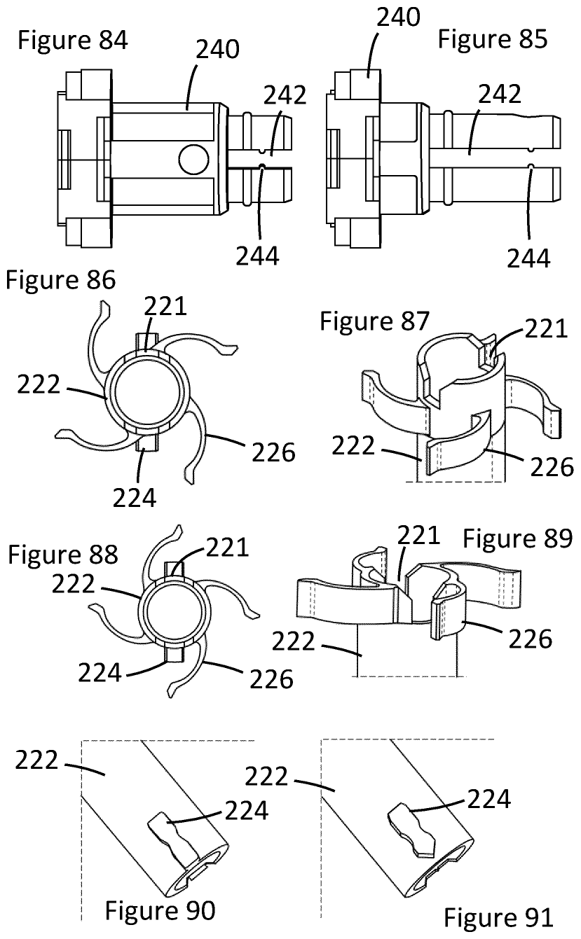

FIGS. 80 to 85 will now be used to describe the powerpack housing 240 in more detail using several different example powerpack housings. In general, the powerpack housing is immovably attached to the housing 40 (specifically the proximal housing 42, where the housing comprises a proximal housing 42 and a distal housing 44). The powerpack housing 240 comprises a slit 242; the protrusion 224 of the powerpack lock 220 is in the slit during part of the assembly process of a medicament delivery device to stop the torsion spring from rotating the powerpack lock 220 relative to the powerpack housing 240 prematurely. The powerpack housing 240 comprises a protrusion 244 of the powerpack housing 240; the protrusion 244 engages a corresponding notch 225 (see FIG. 93, for example) of the protrusion 224 of the powerpack lock 220 to help align the powerpack lock 220 relative to the powerpack housing 240 during part of the assembly process.

The powerpack housing 240 comprises a rib 246. During medicament delivery, the rib 246 engages a corresponding further protrusion 226 of the powerpack lock 220 (FIG. 16), providing feedback (particularly audible feedback) to a user that medicament delivery is ongoing, as the powerpack lock 220 rotates relative to the powerpack housing 240 during medicament delivery. The rib 246 of the powerpack housing 240 can take various shapes, depending for example on the shape of the further protrusion 226 of the powerpack lock 220, with examples shown in FIGS. 80, 81 and 83.

The powerpack housing 240 comprises a cut-out 247. The distal end protrusion 264 of the torsion spring 260 extends through the cut-out 247. As a result, the torsion spring 260 is engaged with the powerpack housing 240 at the distal end of the torsion spring and with the driver 280 at the proximal end of the torsion spring 260.

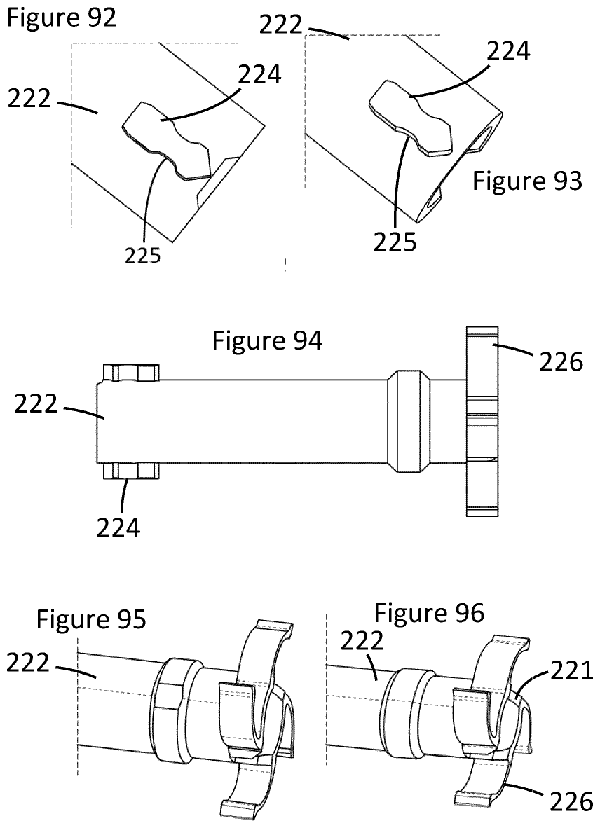

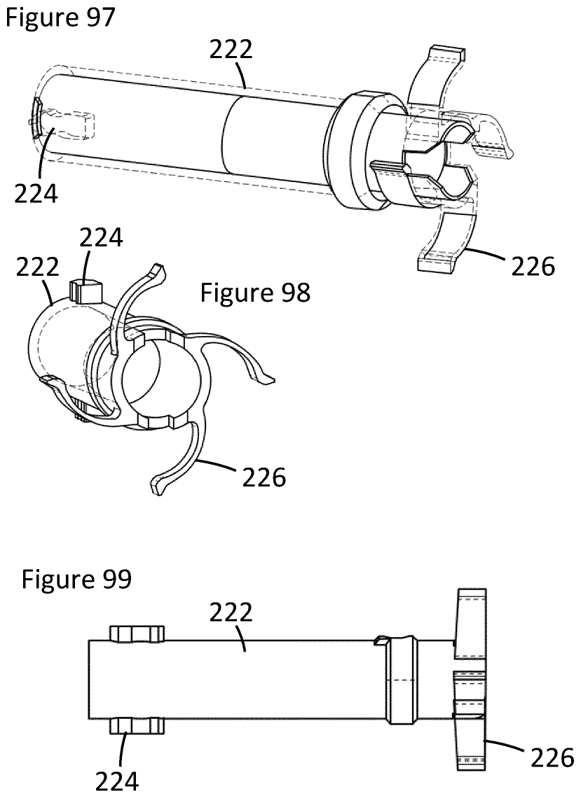

FIGS. 86 to 99 will now be used to describe the powerpack lock 220 in more detail using various different example powerpack locks, along with the powerpack lock in FIG. 16. The powerpack lock 220 comprises a body 222, a protrusion 224 and a further protrusion 226; the protrusion 224 of the powerpack lock 220 engages the powerpack housing 240 and the driver 280 as described above, and the further protrusion 226 engages the powerpack housing 240 as described above. The protrusion 224 and the further protrusion 226 are both attached to the body. The body comprises a notch 221; the notch 221 is at the distal end of the body. In an assembled medicament delivery device, the notch 221 engages with a lock protrusion 381 of the spinner 380 to rotationally lock the powerpack lock 220 and the lock protrusion 381 of the spinner 380, so that the spinner rotates when the powerpack lock rotates during medicament delivery (thereby providing a visual (and/or tactile, if desired) indication to the user that medicament delivery is in progress). Optionally, the protrusion 224 of the powerpack lock 220 is chamfered at the proximal end (e.g. FIG. 91), rather than the proximal end extending in the circumferential direction (e.g. FIG. 90).

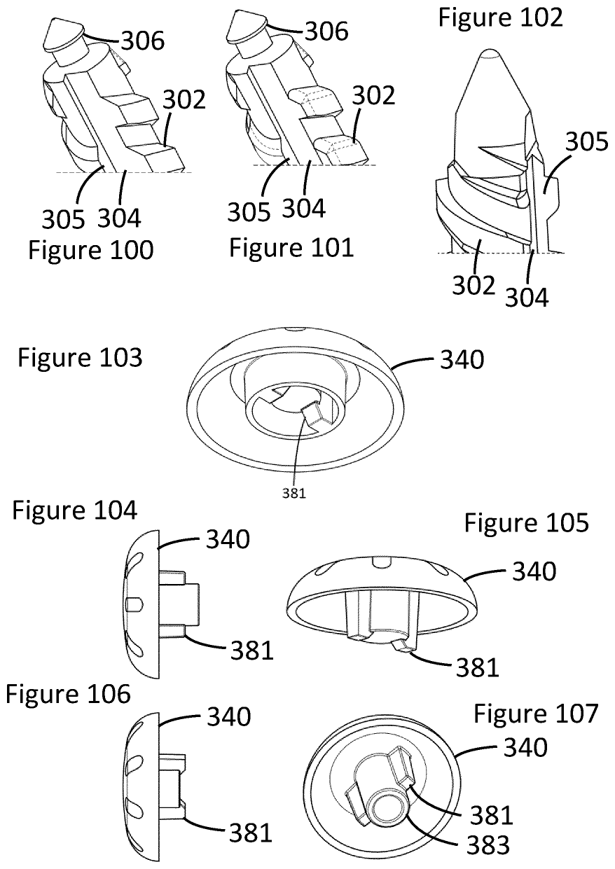

FIGS. 100 to 102 show close-ups of examples of the proximal end (FIGS. 100 and 101) and the distal end (FIG. 102) of a plunger rod such as the plunger rod 300 in FIG. 16. FIGS. 100 and 101 show examples of differently shaped threads on the plunger rod. The plunger rods 300 described herein include two flattened sides 304, but as with the duplicated features herein in general, only one is needed. As previously mentioned, the plunger rod doesn't have to have a flattened side at all, but just needs to be non-circular in cross section where it engages the driver so that it can be rotated by the driver.

The screw thread 302 is typically on a portion of the plunger that does not engage the driver, with the screw thread typically engaged by the lock component 101.

A snap fit ledge 306 is provided to engage snap fit arms 342 of the thrust bearing 340 (or more specifically, to engage inwardly facing protrusions 344 of the thrust bearing 340) (see FIG. 18 in particular). The snap fit ledge 306 is optional and is one example of how the plunger rod could engage a thrust bearing when a thrust bearing is provided; alternatively, the proximal end of the plunger rod could be shaped to instead directly engage a stopper.

FIGS. 103 to 107 will now be used to describe the spinner 380 in more detail using various different example spinners. As described above, the spinner 380 is rotationally locked to the powerpack lock 220 by the interaction of the notch 221 of the powerpack lock 220 with the lock protrusion 381 of the spinner 380 in an assembled medicament delivery device. Optionally, the spinner 380 comprises a central body 383 that extends along the axis (e.g. FIGS. 104, 105 and 107), and optionally the central body 383 is hollow (see FIG. 107). The central body can help align the powerpack lock 220 relative to the spinner 380. In general, the spinner is optional, but can be useful to provide feedback, particularly visual and/or tactile feedback. Optionally, the spinner is an integral part of the powerpack lock 220.

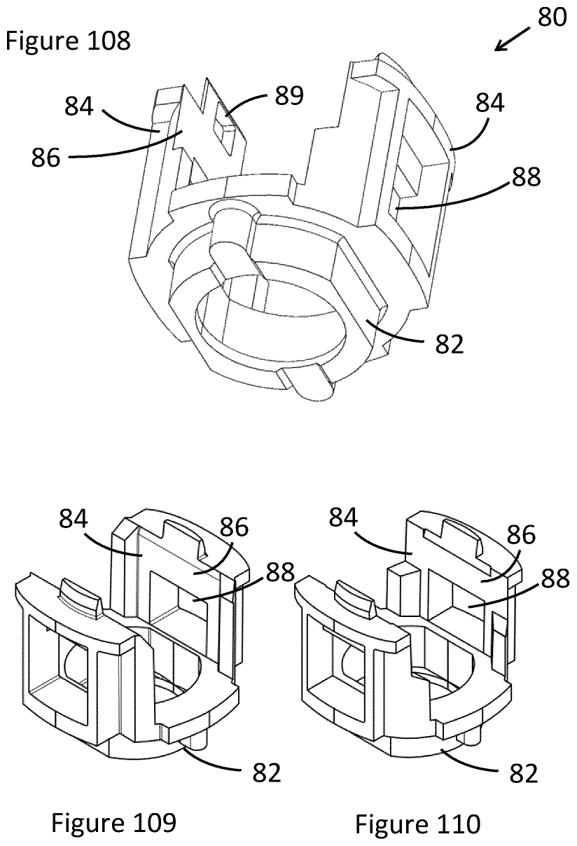

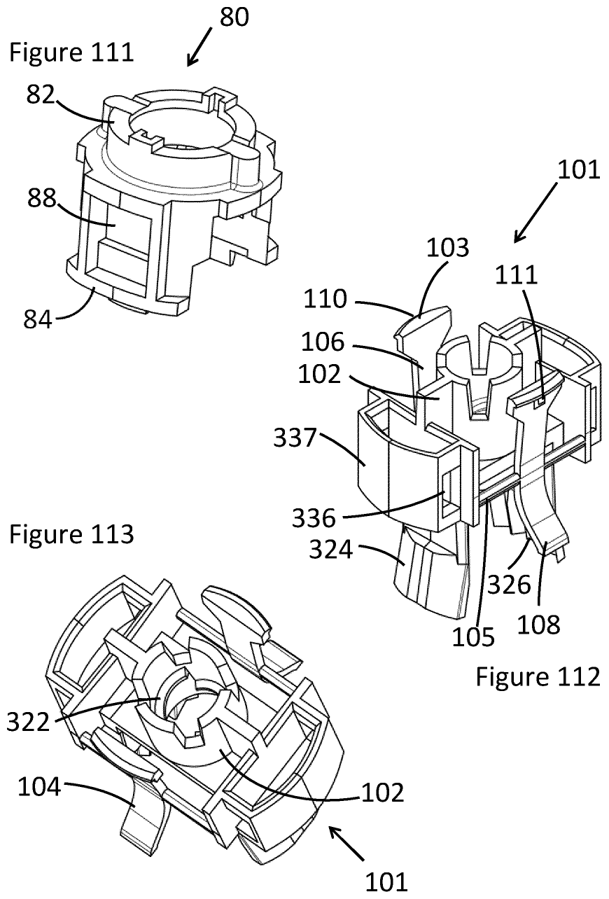

FIGS. 14 and 108 to 111 will now be used to describe the lock activation sleeve 80 in more detail. The lock activation sleeve 80 comprises a base 82 and two arms 84, though one, three or more arms could alternatively be provided. Each arm comprises a cut-out 88, which can accommodate the distal part 108 of the arm of the lock component 101 (see FIG. 8 for example). Each arm comprises a pad 86, which engages the lock component 101 as described above. In some examples (FIGS. 14 and 108), each arm comprises an arm 91 comprising a distally facing surface 89 as described previously. The base 102 and the flexible arm 104 can take various shapes, depending for example of the level of component rigidity desired and the shape of other medicament delivery device components.

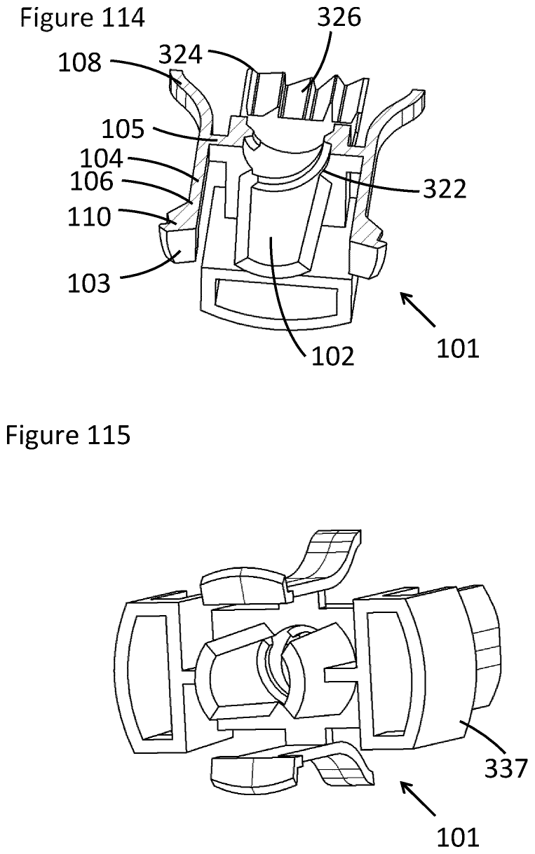

The functionality of the lock component 101 (FIGS. 1 to 7) and the lock activation sleeve 80 (FIG. 14) is described in detail elsewhere in the application. FIGS. 112 and 113 show one alternative lock component design, and FIGS. 114 and 115 show another alternative lock component design. One difference in the example in FIGS. 114 and 115 is the shape of the pivot 105. In this example, the pivot is a (living) hinge, whereas in the example in FIG. 1, the pivot is a rod, with the two ends of the pivot being attached to the base and the middle of the pivot being attached to the flexible arm. In more detail: in general, for the lock component, the activation prevention mechanism works with any sleeve-activated device, and should not need to rely on integration with a seesaw, as only one side of the seesaw is used for the activation prevention. This means a one-sided cantilever beam works as well as a pivot. The beam has a hook that holds the activation sleeve in place and the beam is pushed radially or rotationally out of the way to disengage from the activation sleeve. An alternative to the sleeve being pushed to disengage the hook is therefore that the activation sleeve is the flexible part which disengages from a fully static and non-flexible hook (that is, the proximal part of the flexible arm is optional). It can flex either radially inward, outward, flex rotationally, or move rotationally upon interaction with a needle cover to disengage. One alternative design of a seesaw is one which is not pivoting around beams on either side of the seesaw, but instead supported by a so called “living hinge”, a thin section of plastic around which it can pivot (the example in FIGS. 114 and 115). As with other features of the lock component, which is a relatively complex part, optimisation of the design of the pivot may optionally take into account mouldability of the design, thereby allowing for optimisation of properties such as complexity of moulding tool design and the number of parts that can be moulded before a moulding tool becomes worn and needs replacing.

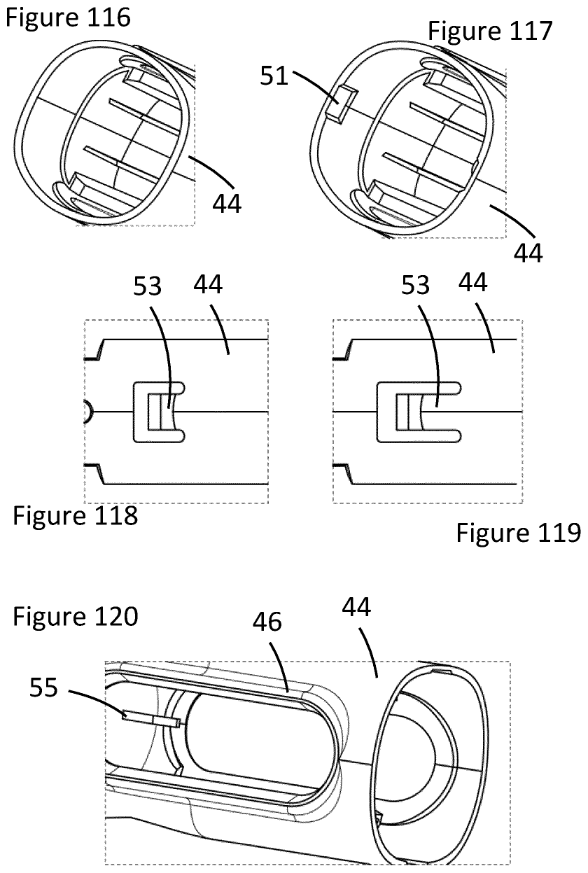

Some details and structural options for the distal housing 44 will now be described in more detail with reference to FIGS. 116 to 124. FIGS. 116 and 117 show two alternatives for the proximal part of the distal housing 44. In FIG. 117, an optional recess 51 is provided at the proximal end of the inner surface of the distal housing 44. If needed, this can provide extra space (or extra tolerance), beyond the space already provided by removal of the cap, to allow the flexible arm 605 of the proximal housing 42 to flex out when the needle guard 60 is moved in the distal direction relative to the proximal housing 42 (see for example FIGS. 45 and 46 for the structure without a recess 51 of the proximal housing 42).

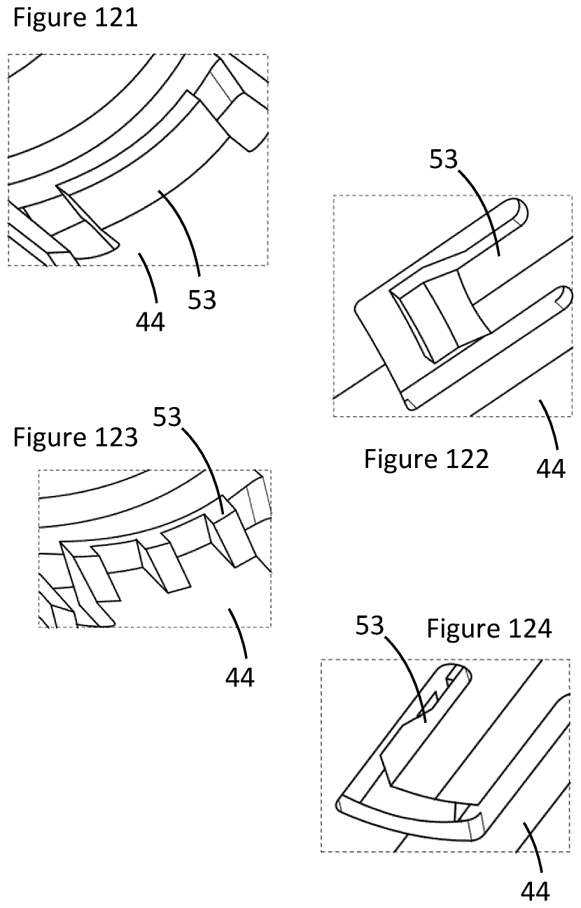

FIGS. 118 and 119 show two examples of an optional snap fit arm 53 that could be provided in the distal housing 44 to help hold the lock component 101 in place relative to the distal housing 44 (for example by engaging with a distally facing surface 338 of the arm 337 of the lock component 101; see FIG. 2). FIGS. 121 to 124 show further details of options for the snap-fit arm 53. In particular, FIG. 124 shows the outer surface of the snap-fit arm 53 being aligned with the outer surface of the rest of the distal housing 44, which can help with supporting the label 419, for example (FIG. 122 shows an alternative where the outer surface of the snap-fit arm 53 is not aligned with the outer surface of the rest of the distal housing 44, but is instead recessed). FIG. 120 shows an optional rib 55 (of the distal housing 44) extending towards the axis from the inner surface of the distal housing 44, which can be provided to help support the syringe holder 120. The rib 55 is adjacent to the window 46 in this example (and arranged distal the window), though the location of the rib could be altered, for example to align with differing syringe holder designs.

Where more than one of a feature is provided in an example given herein (for example two arms, two protrusions, two ribs), in general one or more of the feature could be provided.

Nevertheless, provision of a plurality of the feature (e.g. two, three, four or more) can be beneficial, for example to spread loads. Symmetrical placement of the plurality of features (for example opposite to one another relative to an axis, or equally spread around a circumference) is also optional but can be beneficial to help equally distribute loads.

A number of the components herein do not move relative to each other during medicament delivery device use, but are provided as separate components to help with assembly of and/or making the components (for example to address moulding limitations). However, it is not essential that these components be separate parts, and it is not essential that these components be moulded (for example, they could be made by additive manufacturing instead). As such, two or more of these components, such as the housing components (proximal housing 42, distal housing 44, powerpack housing 240), the syringe holder and the lock component (the base of which normally does not move relative to the housing during medicament delivery), could be combined together as a single integral part. For example, the proximal housing 42 and the distal housing 44 could be a single integral part. Many of the components described herein are cylindrical in the depicted examples. Typically, the cylinders have a circular cross section, although this is not essential.

In this application, examples have been described with an emphasis on autoinjectors with needles. However, the concepts described herein could also be implemented in pen injectors or medicament delivery devices more generally. Instead of a needle, another medicament delivery member could be used, such as a jet injector, for example. An autoinjector is generally defined as an injection device in which at least one part of the process, for example medicament injection, needle insertion or needle guard retraction, is carried out by the autoinjector and therefore does not need to be carried out by the user. A powerpack (or rear sub-assembly) of a medicament delivery device is generally defined as the part of the device that holds a power source such as a spring, a gas canister or a battery. In the example in FIG. 13, the cap, the needle guard 60, the needle guard spring 140, the syringe holder 120, the proximal housing 42 and the syringe 362 are not part of the powerpack, but the other components shown are part of the powerpack. The cap, the needle guard 60, the needle guard spring 140, the syringe holder 120 and the proximal housing 42 make up a front sub-assembly.

The lock activation sleeve 80 can be different shapes depending on the device and the functionality desired. In the examples described above, the lock activation sleeve 80 comprises a base 82 (which is tubular in this example), arms 84 with pads 86 and cut-outs 88 for the distal part 108 of the arm of the needle guard lock 100. Recesses could be provided instead of cut-outs. In general, the arms are also optional and the pads 86 could be on the base 82; the functional reason for the arms 84 of the lock activation sleeve is that it allows the arms 324 of the lock component 101 to extend outwards radially without the base 82 of the lock activation sleeve getting in the way. The lock activation sleeve is typically arranged in the housing at the distal end of the needle guard lock, with most or all of the lock activation sleeve further from the proximal end than the needle guard prior to device use. The shape of the pads 86 can vary considerably and still provide a functional lock activation sleeve. The primary requirement is that the pad pushes the distal part 108 of the arm 104 towards the axis when the lock activation sleeve is moved in the distal direction, and as such, the pad can simply be a protrusion on the arm 84 of the lock activation sleeve or even a flat surface of the arm 84 of the lock activation sleeve.

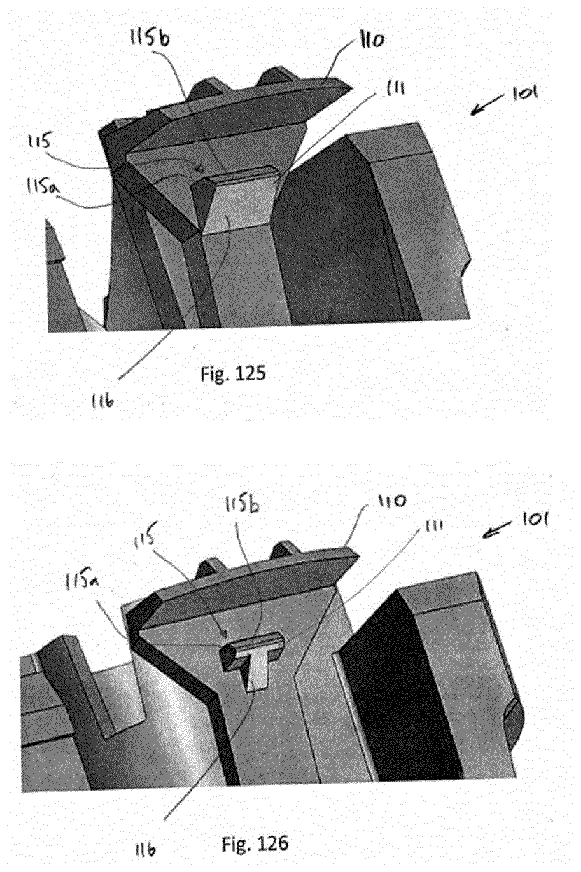

Referring to FIG. 125, a further embodiment of the lock component 101 is identical to the embodiment of the lock component 101 described with reference to FIGS. 1 to 7, except that the protrusion 111 of the further embodiment is modified as now described. In more detail, in the further embodiment, the proximally facing surface 115 provided on the protrusion 111 has a first portion 115a and a second portion 115b. The second portion 115b is located radially outward of the first portion 115a. That is, the second portion 115b is further from the axis 20 than the first portion 115a. Both the first portion 115a and the second portion 115b face proximally. Both the first portion 115a and the second portion 115b are substantially planar, although in some embodiments they may be curved and/or chamfered at their edges.

Referring to FIG. 126, yet a further embodiment of the lock component 101 is identical to the embodiment of the lock component 101 described with reference to FIGS. 125, with the protrusion 111 again being modified such that the proximally facing surface 115 provided on the protrusion 111 has a first portion 115a and a second portion 115b. The second portion 115b is again located radially outward of the first portion 115a. That is, the second portion 115b is further from the axis 20 than the first portion 115a. Both the first portion 115a and the second portion 115b face proximally. However, in this embodiment, the protrusion 111 is generally narrower in a circumferential direction. A distally facing surface 116 of the protrusion 111 is narrower than the proximally facing surface 115. That is, the distally extending surface 116 ends less in a circumferential direction relative to the axis 20 then the proximally extending surface 115. This distally extending surface 116 is sloped radially outwards. This assists interaction between the protrusion 111 and the activation sleeve 80, as previously described.

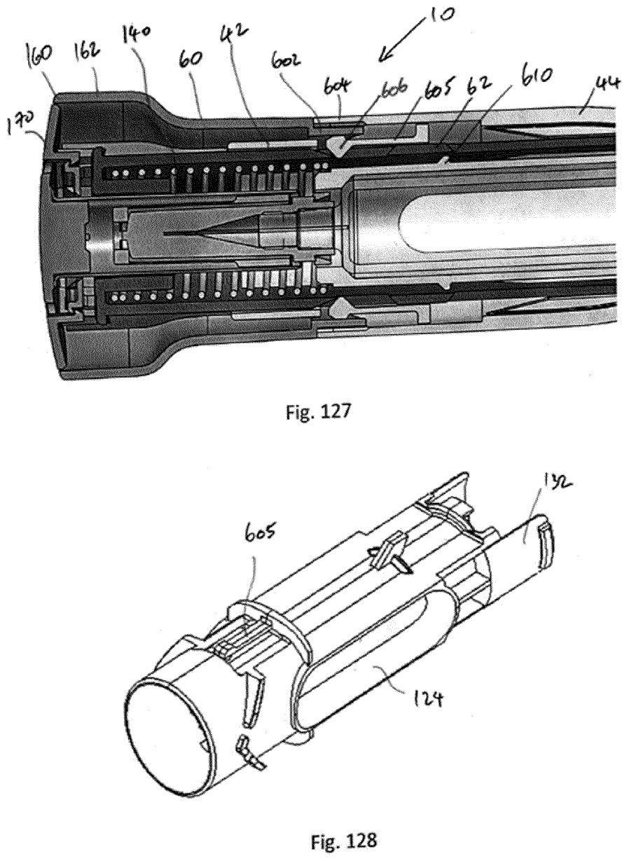

Referring to FIG. 127, a further embodiment of the medicament delivery device 10 is similar to that described with reference to FIGS. 12 to 25. However, in the embodiment shown in FIG. 127, the protrusion 610 of the syringe holder 120 is located distally of the protrusion 606 of the flexible arm 605 of the housing 44. Also, the housing 44 and the syringe holder 120 are a single integral piece, as shown in FIG. 128.

The delivery devices described herein can be used for the treatment and/or prophylaxis of one or more of many different types of disorders.

Exemplary disorders include, but are not limited to: rheumatoid arthritis, inflammatory bowel diseases (e.g. Crohn's disease and ulcerative colitis), hypercholesterolaemia and/or dyslipidemia, cardiovascular disease, diabetes (e.g. type 1 or 2 diabetes), psoriasis, psoriatic arthritis, spondyloarthritis, hidradenitis suppurativa, Sjögren's syndrome, migraine, cluster headache, multiple sclerosis, neuromyelitis optica spectrum disorder, anaemia, thalassemia, paroxysmal nocturnal hemoglobinuria, hemolytic anaemia, hereditary angioedema, systemic lupus erythematosus, lupus nephritis, myasthenia gravis, Behçet's disease, hemophagocytic lymphohistiocytosis, atopic dermatitis, retinal diseases (e.g., age-related macular degeneration, diabetic macular edema), uveitis, infectious diseases, bone diseases (e.g., osteoporosis, osteopenia), asthma, chronic obstructive pulmonary disease, thyroid eye disease, nasal polyps, transplant, acute hypoglycaemia, obesity, anaphylaxis, allergies, sickle cell disease, Alzheimer's disease, Parkinson's disease, dementia with Lewy bodies, systemic infusion reactions, immunoglobulin E (IgE)-mediated hypersensitivity reactions, cytokine release syndrome, immune deficiencies (e.g., primary immunodeficiency, chronic inflammatory demyelinating polyneuropathy), enzyme deficiencies (e.g., Pompe disease, Fabry disease, Gaucher disease), growth factor deficiencies, hormone deficiencies, coagulation disorders (e.g., hemophilia, von Willebrand disease, Factor V Leiden), and cancer.

Exemplary types of drugs that could be included in the delivery devices described herein include, but are not limited to, small molecules, hormones, cytokines, blood products, enzymes, vaccines, anticoagulants, immunosuppressants, antibodies, antibody-drug conjugates, neutralizing antibodies, reversal agents, radioligand therapies, radioisotopes and/or nuclear medicines, diagnostic agents, bispecific antibodies, proteins, fusion proteins, peptibodies, polypeptides, pegylated proteins, protein fragments, nucleotides, protein analogues, protein variants, protein precursors, protein derivatives, chimeric antigen receptor T cell therapies, cell or gene therapies, oncolytic viruses, or immunotherapies.

Exemplary drugs that could be included in the delivery devices described herein include, but are not limited to, immuno-oncology or bio-oncology medications such as immune checkpoints, cytokines, chemokines, clusters of differentiation, interleukins, integrins, growth factors, coagulation factors, enzymes, enzyme inhibitors, retinoids, steroids, signaling proteins, pro-apoptotic proteins, anti-apoptotic proteins, T-cell receptors, B-cell receptors, or costimulatory proteins.

Exemplary drugs that could be included in the delivery devices described herein include, but are not limited to, those exhibiting a proposed mechanism of action, such as human epidermal growth factor receptor 2 (HER-2) receptor modulators, interleukin (IL) modulators, interferon (IFN) modulators, complement modulators, glucagon-like peptide-1 (GLP-1) modulators, glucose-dependent insulinotropic polypeptide (GIP) modulators, cluster of differentiation 38 (CD38) modulators, cluster of differentiation 22 (CD22) modulators, C1 esterase modulators, bradykinin modulators, C-C chemokine receptor type 4 (CCR4) modulators, vascular endothelial growth factor (VEGF) modulators, B-cell activating factor (BAFF), P-selectin modulators, neonatal Fc receptor (FcRn) modulators, calcitonin gene-related peptide (CGRP) modulators, epidermal growth factor receptor (EGFR) modulators, cluster of differentiation 79B (CD79B) modulators, tumor-associated calcium signal transducer 2 (Trop-2) modulators, cluster of differentiation 52 (CD52) modulators, B-cell maturation antigen (BCMA) modulators, enzyme modulators, platelet-derived growth factor receptor A (PDGFRA) modulators, cluster of differentiation 319 (CD319 or SLAMF7) modulators, programmed cell death protein 1 and programmed death-ligand 1 (PD-1/PD-L1) inhibitors/modulators, B-lymphocyte antigen cluster of differentiation 19 (CD19) inhibitors, B-lymphocyte antigen cluster of differentiation 20 (CD20) modulators, cluster of differentiation 3 (CD3) modulators, cytotoxic T-lymphocyte-associated protein 4 (CTLA-4) inhibitors, T-cell immunoglobulin and mucin-domain containing-3 (TIM-3) modulators, T cell immunoreceptor with Ig and ITIM domains (TIGIT) modulators, V-domain Ig suppressor of T cell activation (VISTA) modulators, indoleamine 2,3-dioxygenase (IDO or INDO) modulators, poliovirus receptor-related immunoglobulin domain-containing protein (PVRIG) modulators, lymphocyte-activation gene 3 (LAG3; also known as cluster of differentiation 223 or CD223) antagonists, cluster of differentiation 276 (CD276 or B7-H3) antigen modulators, cluster of differentiation 47 (CD47) antagonists, cluster of differentiation 30 (CD30) modulators, cluster of differentiation 73 (CD73) modulators, cluster of differentiation 66 (CD66) modulators, cluster of differentiation w137 (CDw137) agonists, cluster of differentiation 158 (CD158) modulators, cluster of differentiation 27 (CD27) modulators, cluster of differentiation 58 (CD58) modulators, cluster of differentiation 80 (CD80) modulators, cluster of differentiation 33 (CD33) modulators, cluster of differentiation 159 (CD159 or NKG2) modulators, glucocorticoid-induced TNFR-related (GITR) protein modulators, Killer Ig-like receptor (KIR) modulators, growth arrest-specific protein 6 (GAS6)/AXL pathway modulators, A proliferation-inducing ligand (APRIL) receptor modulators, human leukocyte antigen (HLA) modulators, epidermal growth factor receptor (EGFR) modulators, B-lymphocyte cell adhesion molecule modulators, cluster of differentiation w123 (CDW123) modulators, Erbb2 tyrosine kinase receptor modulators, endoglin modulators, mucin modulators, mesothelin modulators, hepatitis A virus cellular receptor 2 (HAVCR2) antagonists, cancer-testis antigen (CTA) modulators, tumor necrosis factor receptor superfamily, member 4 (TNFRSF4 or OX40) modulators, adenosine receptor modulators, inducible T cell co-stimulator (ICOS) modulators, cluster of differentiation 40 (CD40) modulators, tumor-infiltrating lymphocytes (TIL) therapies, or T-cell receptor (TCR) therapies.

Exemplary drugs that could be included in the delivery devices described herein include, but are not limited to: etanercept, abatacept, adalimumab, evolocumab, exenatide, secukinumab, erenumab, galcanezumab, fremanezumab-vfrm, alirocumab, methotrexate (amethopterin), tocilizumab, interferon beta-1a, interferon beta-1b, peginterferon beta-1a, sumatriptan, darbepoetin alfa, belimumab, sarilumab, semaglutide, dupilumab, reslizumab, omalizumab, glucagon, epinephrine, naloxone, insulin, amylin, vedolizumab, eculizumab, ravulizumab, crizanlizumab-tmca, certolizumab pegol, satralizumab, denosumab, romosozumab, benralizumab, emicizumab, tildrakizumab, ocrelizumab, ofatumumab, natalizumab, mepolizumab, risankizumab-rzaa, ixekizumab, and immune globulins.

Exemplary drugs that could be included in the delivery devices described herein may also include, but are not limited to, oncology treatments such as ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab.

Exemplary drugs that could be included in the delivery devices described herein include “generic” or biosimilar equivalents of any of the foregoing, and the foregoing molecular names should not be construed as limiting to the “innovator” or “branded” version of each, as in the non-limiting example of innovator medicament adalimumab and biosimilars such as adalimumab-afzb, adalimumab-atto, adalimumab-adbm, and adalimumab-adaz.

Exemplary drugs that could be included in the delivery devices described herein also include, but are not limited to, those used for adjuvant or neoadjuvant chemotherapy, such as an alkylating agent, plant alkaloid, antitumor antibiotic, antimetabolite, or topoisomerase inhibitor, enzyme, retinoid, or corticosteroid. Exemplary chemotherapy drugs include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Exemplary drugs that could be included in the delivery devices described herein also include, but are not limited to, analgesics (e.g., acetaminophen), antipyretics, corticosteroids (e.g. hydrocortisone, dexamethasone, or methylprednisolone), antihistamines (e.g., diphenhydramine or famotidine), antiemetics (e.g., ondansetron), antibiotics, antiseptics, anticoagulants, fibrinolytics (e.g., recombinant tissue plasminogen activator [r-TPA]), antithrombolytics, or diluents such as sterile water for injection (SWFI), 0.9% Normal Saline, 0.45% normal saline, 5% dextrose in water, 5% dextrose in 0.45% normal saline, Lactated Ringer's solution, Heparin Lock Flush solution, 100 U/mL Heparin Lock Flush Solution, or 5000 U/mL Heparin Lock Flush Solution.

Pharmaceutical formulations including, but not limited to, any drug described herein are also contemplated for use in the delivery devices described herein, for example pharmaceutical formulations comprising a drug as listed herein (or a pharmaceutically acceptable salt of the drug) and a pharmaceutically acceptable carrier. Such formulations may include one or more other active ingredients (e.g., as a combination of one or more active drugs), or may be the only active ingredient present, and may also include separately administered or co-formulated dispersion enhancers (e.g. an animal-derived, human-derived, or recombinant hyaluronidase enzyme), concentration modifiers or enhancers, stabilizers, buffers, or other excipients.

Exemplary drugs that could be included in the delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOpAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Various modifications to the embodiments described are possible and will occur to those skilled in the art without departing from the invention which is defined by the following claims.

Some aspects of the disclosure are summarised as follows.
1. A lock component for a medicament delivery device, the lock component extending along an axis from a proximal end to a distal end, the lock component comprising a base, a pivot and a flexible arm,
   wherein the flexible arm is attached to the base by the pivot,
   wherein the flexible arm comprises a proximal part extending proximally from the pivot.
   wherein the flexible arm is configured to support accidental activation prevention in a medicament delivery device, and
   wherein the flexible arm is configured to support medicament delivery member lockout in a medicament delivery device.
2. The lock component of clause 1, wherein the flexible arm comprises a first proximally facing surface that is configured to engage a distally facing surface of another medicament delivery device component so as to provide accidental activation prevention in a medicament delivery device.
3. The lock component of clause 1 or 2, wherein the lock component comprises a first protrusion, and the first proximally facing surface is on the first protrusion.
4. The lock component of clause 2 or 3, wherein the proximal part of the flexible arm comprises the first proximally facing surface.
5. The lock component of clause 3 or 4, wherein the first protrusion extends away from the axis.
6. The lock component of clause 5, wherein the proximally facing surface extends perpendicular to the axis.
7. The lock component of any of clauses 1 to 6, wherein the flexible arm comprises a second proximally facing surface that is configured to engage a distally facing surface of another medicament delivery device component so as to provide a medicament delivery member guard lockout after medicament delivery device use.
8. The lock component of clause 7, wherein the flexible arm comprises a second protrusion, and wherein the second protrusion comprises the second proximally facing surface.
9. The lock component of clause 8, wherein the second protrusion extends from the proximal part of the flexible arm.
10. The lock component of clause 8 or 9, wherein the second protrusion extends away from the axis relative to the rest of the proximal part of the flexible arm.
11. The lock component of any of clauses 7 to 10, wherein the second proximally facing surface comprises a notch that is configured to engage said distally facing surface of another medicament delivery device component so as to provide a medicament delivery member guard lockout after medicament delivery device use.
12. The lock component of clause 11, wherein the notch is v- or u-shaped.
13. The lock component of any of clauses 8 to 12, wherein the second protrusion is the proximal-most part of the proximal part of the flexible arm.
14. The lock component of any of clauses 3 to 6 in combination with any of clauses 8 to 13, wherein the first protrusion is aligned in the axial direction with the second protrusion.
15. The lock component of clause 14, wherein the first protrusion is spaced apart from the second protrusion.
16. The lock component of clause 14 or 15, wherein the first protrusion is arranged distal to the second protrusion.
17. The lock component of any previous clause, wherein the lock component comprises a snap-fit hole (336) to attach the lock component to a medicament delivery container holder (120).
18. The lock component of any previous clause, wherein the lock component comprises an arm (337) to lock the lock component to a housing.
19. The lock component of any previous clause, wherein the lock component comprises either a screw thread (322) or a protrusion, so as to engage a corresponding screw thread on a plunger rod.
20. The lock component of any previous clause, wherein the lock component comprises a lock component arm (324), the lock component arm being flexible and comprising at least one tooth (286) to engage a corresponding tooth on a driver of a medicament delivery device so as to limit rotation of the driver relative to the lock component prior to medicament delivery.
21. The lock component of any previous clause, wherein the flexible arm is configured to support accidental activation prevention in a medicament delivery device by engaging a proximally facing surface of a lock activation sleeve of the medicament delivery device, thereby blocking distal movement of the lock activation sleeve relative to the flexible arm so as to stop medicament delivery device activation.
22. The lock component of any previous clause, wherein the flexible arm is configured to support medicament delivery member lockout in a medicament delivery device by engaging a recess or slit in a housing of a medicament delivery device when a medicament delivery member guard of the medicament delivery device is pushed against the flexible arm, thereby blocking distal movement of the medicament delivery member guard relative to the flexible arm.
23. The lock component of any previous clause, wherein the flexible arm comprises a distal part extending distally from the pivot.
24. The lock component of any previous clause, wherein the lock component is a single integral part.
25. A medicament delivery device sub-assembly comprising a lock activation sleeve and the lock component of any of clauses 2 to 24 when dependent on clause 2, wherein the another medicament delivery device component of clause 2 is the lock activation sleeve.
26. The medicament delivery device sub-assembly of clause 25, wherein the lock activation sleeve comprises a pad (86).
27. The medicament delivery device sub-assembly of clause 26, wherein the pad is adjacent to the distal part of the flexible arm of the lock component.
28. The medicament delivery device sub-assembly of clause 26 or 27, wherein the lock activation sleeve comprises a base (82) and an arm (84) extending from the base, and wherein the pad (86) is an inwardly facing surface of the arm (84).
29. The medicament delivery device sub-assembly of any of clauses 25 to 28, wherein the distally facing surface (89) is in a recess or cut-out in the arm (84).wherein the lock activation sleeve extends around the lock component.
30. The medicament delivery device sub-assembly of any of clauses 25 to 30, wherein the sub-assembly comprises a medicament delivery member guard, and wherein a distally facing surface of the medicament delivery member guard is configured to engage a corresponding proximally facing surface of the lock activation sleeve so as to move the lock activation sleeve in a distal direction relative to the lock component, thereby allowing medicament delivery.
31. A medicament delivery device sub-assembly, wherein the sub-assembly comprises a housing and a lock component according to any of clauses 7 to 24, and wherein the another medicament delivery device component of clause 7 is the housing.
32. The medicament delivery device sub-assembly of clause 32, wherein the housing comprises a recess or cut-out (45), and wherein the second protrusion is at least partially inside the recess or cut-out (45) after the medicament delivery device is used.
33. The medicament delivery device sub-assembly of clause 32 or 33, wherein the lock component is inside the housing.
34. A medicament delivery device sub-assembly according to any of clauses 25 to 33, wherein the sub-assembly comprises a medicament delivery container holder (120).
35. A medicament delivery device sub-assembly according to any of clauses 25 to 34, wherein the sub-assembly comprises a driver.
36. A medicament delivery device sub-assembly according to any of clauses 25 to 35, wherein the sub-assembly comprises a plunger rod.
37. A medicament delivery device powerpack comprising the lock component of any of clauses 1 to 24 or the sub-assembly of any of clauses 25 to 37.
38. A medicament delivery device comprising the lock component of any of clauses 1 to 24 or the medicament delivery device sub-assembly of any of clauses 25 to 37 or the powerpack of clause 38.
39. The medicament delivery device of clause 39, wherein the medicament delivery device is an autoinjector.
40. A medicament delivery device component, the component comprising a first flexible arm, the first flexible arm being configured to prevent accidental activation of a medicament delivery device and the first flexible arm being configured to provide a lockout after use of said medicament delivery device so that a medicament delivery member of said medicament delivery device is protected.

Further aspects of the disclosure are summarised as follows.
1. A sub-assembly for a medicament delivery device, the sub-assembly extending along an axis from a proximal end to a distal end, the sub-assembly comprising:
   a housing, the housing being tubular and comprising a flexible arm, the flexible arm comprising a protrusion extending towards the axis and a surface facing away from the axis;
   a cap, the cap being removably attached to a proximal end of the housing, the cap comprising a surface facing towards the axis;
   a syringe holder attached to the housing, the syringe holder comprising a protrusion extending away from the axis;
   a medicament delivery member guard arranged telescopically at least partially inside the housing, the medicament delivery member guard comprising a recess or cut-out;
wherein the surface of the cap is adjacent to the surface of the housing to restrict movement of the flexible arm away from the axis when the cap is attached to the housing;
wherein the protrusion of the flexible arm (and/or the protrusion of the syringe holder) is in the recess or cut-out of the medicament delivery member guard; and wherein the protrusion of the syringe holder is adjacent to (and/or aligned in a radial direction relative to the axis with) the protrusion of the flexible arm of the housing, so that, when the cap is attached to the housing, the protrusion of the flexible arm of the housing and the protrusion of the syringe holder together restrict distal movement of the medicament delivery member guard relative to the housing.
2. The sub-assembly of clause 1, wherein the flexible arm of the housing comprises an outwardly extending protrusion, and the outwardly extending protrusion comprises the surface facing away from the axis.
3. The sub-assembly of clause 1 or 2, wherein the cap comprises a protrusion, and the surface facing towards the axis is on the protrusion.
4. The sub-assembly of clause 3, wherein the cap comprises a surface facing away from the axis; wherein the surface facing away from the axis is adjacent to an inwardly facing surface of the housing, thereby restricting movement of the protrusion of the cap away from the axis.
5. The sub-assembly of any previous clause, wherein the syringe holder is tubular.
6. The sub-assembly of any previous clause, wherein the sub-assembly comprises a medicament delivery member guard spring arranged between the housing and the medicament delivery member guard to bias the medicament delivery member guard in the proximal direction relative to the housing.
7. The sub-assembly of any previous clause, wherein the housing comprises a proximal housing and a distal housing, and the proximal housing and the distal housing are two separate components.
8. The sub-assembly of clause 7 when dependent on any combination of clauses that includes clause 4, wherein the distal housing comprises the inwardly facing surface of the housing and the proximal housing comprises the flexible arm of the housing.
9. The sub-assembly of any previous clause, wherein the cap comprises a medicament delivery member shield remover.
10. The sub-assembly of clause 9, wherein the cap comprises the medicament delivery member shield remover, a cap housing and a cap insert, and the medicament delivery member shield remover is arranged between the cap housing and the cap insert.
11. The sub-assembly of any previous clause, wherein an inner surface of the housing comprises a recess that can receive the flexible arm after the cap has been removed from the sub-assembly.
12. A medicament delivery device comprising the sub-assembly of any previous clause.
13. The sub-assembly of any previous clause, wherein the recess or cut-out is a recess.
14. The sub-assembly of clause 13, wherein the recess is in the outer surface of the medicament delivery member guard, and the protrusion of the flexible arm is in the recess of the medicament delivery member guard.
15. An autoinjector comprising the sub-assembly of any of clauses 1 to 14.

1-15. (canceled)

16. A sub-assembly for a medicament delivery device, the sub-assembly extending along an axis from a proximal end to a distal end, the sub-assembly comprising:
a housing, the housing being tubular and comprising a flexible arm, the flexible arm comprising a protrusion extending towards the axis and a surface facing away from the axis;
a cap, the cap being removably attached to a proximal end of the housing, the cap comprising a surface facing towards the axis;
a syringe holder attached to the housing, the syringe holder comprising a protrusion extending away from the axis; and
a medicament delivery member guard arranged telescopically at least partially inside the housing, the medicament delivery member guard comprising a recess or cut-out,
wherein the surface of the cap is adjacent to the surface of the housing to restrict movement of the flexible arm away from the axis when the cap is attached to the housing,
wherein the protrusion of the flexible arm is in the recess or cut-out of the medicament delivery member guard, and
wherein, when the cap is attached to the housing, the protrusion of the flexible arm of the housing and the protrusion of the syringe holder together restrict distal movement of the medicament delivery member guard relative to the housing.

17. The sub-assembly of claim 16, wherein the protrusion of the syringe holder is adjacent to the protrusion of the flexible arm of the housing.

18. The sub-assembly of claim 16, wherein the protrusion of the syringe holder is located distally of the protrusion of the flexible arm of the housing.

19. The sub-assembly of claim 16, wherein the housing and the syringe holder are a single integral piece.

20. The sub-assembly of claim 16, wherein the flexible arm of the housing comprises an outwardly extending protrusion, and the outwardly extending protrusion comprises the surface facing away from the axis.

21. The sub-assembly of claim 16, wherein the cap comprises a protrusion, and the surface facing towards the axis is on the protrusion.

22. The sub-assembly of claim 20, wherein the cap comprises a surface facing away from the axis, wherein the surface facing away from the axis is adjacent to an inwardly facing surface of the housing, thereby restricting movement of the protrusion of the cap away from the axis.

23. The sub-assembly of claim 16, wherein the syringe holder is tubular.

24. The sub-assembly of claim 16, wherein the sub-assembly comprises a medicament delivery member guard spring arranged between the housing and the medicament delivery member guard to bias the medicament delivery member guard in the proximal direction relative to the housing.

25. The sub-assembly of claim 16, wherein the housing comprises a proximal housing and a distal housing, and the proximal housing and the distal housing are two separate components.

26. The sub-assembly of claim 24, wherein the distal housing comprises the inwardly facing surface of the housing and the proximal housing comprises the flexible arm of the housing.

27. The sub-assembly of claim 16, wherein the cap comprises a medicament delivery member shield remover.

28. The sub-assembly of claim 24, wherein the cap comprises the medicament delivery member shield remover, a cap housing, and a cap insert, and the medicament delivery member shield remover is arranged between the cap housing and the cap insert.

29. The sub-assembly of claim 16, wherein an inner surface of the housing comprises a housing recess that can receive the flexible arm after the cap has been removed from the sub-assembly.

30. The sub-assembly of claim 16, wherein the recess or cut-out is a recess.

31. A medicament delivery device comprising the sub-assembly of claim 16.
